(19) United States
(12) Patent Application Publication
(10) Pub. No.: US 2025/0000800 A1
(43) Pub. Date: Jan. 2, 2025

(54) METHODS OF PREPARING DOSAGE FORMS USING 3D PRINTING CONTAINING AMORPHOUS SOLID DISPERSIONS

(71) Applicant: BOARD OF REGENTS, THE UNIVERSITY OF TEXAS SYSTEM, Austin, TX (US)

(72) Inventors: Mo MANIRUZZAMAN, Pflugerville, TX (US); Robert O. WILLIAMS, III, Austin, TX (US); Daniel A. DAVIS, Jr., Austin, TX (US); Rishi THAKKAR, Austin, TX (US)

(21) Appl. No.: 18/708,717

(22) PCT Filed: Nov. 9, 2022
(86) PCT No.: PCT/US2022/079565

§ 371 Date: May 9, 2024

Related U.S. Application Data
(60) Provisional application No. 63/277,387, filed on Nov. 9, 2021.

(51) Int. Cl. A61K 9/16; A61K 31/444; B29B 13/10; B29C 64/314; B29K 105/00; B29K 105/16; B29K 509/00; B33Y 10/00; B33Y 70/00

(52) U.S. Cl. CPC A61K 9/1694; A61K 9/1611; A61K 9/1617; A61K 9/1652; A61K 31/444; B29B 13/10; B29C 64/314; B33Y 70/00; B29K 2105/0032; B29K 2105/0035; B29K 2105/16; B29K 2509/00; B29K 2995/0094; B33Y 10/00

(57) ABSTRACT

The present disclosure provides methods of preparing pharmaceutical forms using an additive manufacturing techniques to create a final dosing form and the active pharmaceutical ingredient is present as an amorphous solid dispersion. These methods comprise using a preformed amorphous solid dispersion in an additive manufacturing method that results in a composition even after the additional energy has been added that retains its amorphous nature.

This application claims the benefit of priority to U.S. Provisional Application No. 63/277,387, filed on Nov. 9, 2021, the entire contents of which are hereby incorporated by reference.

### BACKGROUND

### 1. Field

The present disclosure relates generally to the field of pharmaceuticals and pharmaceutical manufacture. More particularly, it concerns compositions and methods of preparing a pharmaceutical composition as amorphous solid dispersions through additive manufacturing techniques.

### 2. Description of Related Art

A significant number of molecules developed in the pharmaceutical drug discovery and lead optimization process are eliminated due to their dose-dependent poor water solubility and thereby low bioavailability. Many of the marketed drug substances also suffer from poor aqueous solubility and thereby fall under the class II and IV of the biopharmaceutical classification system (BCS), which means that the highest available dose of the drug is insoluble in 250 mL of simulated gastric/intestinal fluid. The pharmaceutical industry has adapted amorphous solid dispersions (ASDs) as a viable formulation technique to overcome these issues. Thermodynamically, a drug in an amorphous state has a higher chemical potential as compared to the crystalline state and thereby amorphous state has a higher reactivity and it depicts an enhanced solubility than the crystalline state which is relatively more stable. Although pure amorphous drugs have a solubility advantage over the crystalline species, they are extremely unstable because of their enhanced reactivity and hence tend to recrystallize and/or form hydrates and solvates by trapping water or other solvents in their lattice, this might lead to degradation and/or altered therapeutic activity of the drug. Considering this, formulations or processes leading to partial amorphous conversions or suffering recrystallization in the biological system would have an unpredictable release, absorption, and thereby bioavailability. Hence, these type of formulations cannot be considered as a viable pharmaceutical dosage form. This phenomenon of partial amorphous conversion has been observed in multiple selective laser sintering based 3D printed dosage forms, reported previously in the literature. Even though the partial amorphous conversion was observed in these examples, the phenomenon was not intended or controlled by manipulating the processing parameters. Moreover, the state of the amorphous drug was not investigated as to whether it formed an ASD or merely drug in amorphous form.

ASDs stabilize the amorphous drug by dispersing it in the polymeric matrix. This prevents the drug from recrystallizing. Moreover, the polymer controls the release of the amorphous drug from its matrix, this ensures the controlled release of the drug from the polymeric matrix, which prevents the recrystallization of the drug in the biological system. The two most commonly utilized methods for the preparation of ASDs include hot-melt extrusion (HME) and spray drying (SD), which are used for the majority of drugs, but they have significant limitations. Innovative techniques (thin-film freezing, TFF; KinetiSol Processing, KSD; and micro precipitated bulk powder, MBP) have been developed as viable alternatives to creating ASDs.

Selective Laser Sintering Three-Dimensional Printing (SLS-3DP) is emerging as a viable method to produce pharmaceutical tablets. Research has been dedicated to showing the dynamic applications of this process to the pharmaceutical fields. Specifically, SLS-3DP has been able to highlight its ability to create patient-tailored medications by modification of the printing parameters. The prior art has shown the ability to control the drug release from the tablet matrix by using the highly precise laser to configure different lattice structures. These structures have the ability to control drug release by altering the surface area of the tablet that is exposed to the media.

Unfortunately, the use of SLS and other additive manufacturing methods are generally not applicable to use with amorphous formulations like ADS as the added energy to form the additive manufacturing method results in crystallization of the active pharmaceutical ingredient. Since the amount of energy required to sinter the composition generally is above the glass transition, this energy generally would result in conversion of the active agents into a crystalline form. Therefore, there remains a desire to develop SLS-3DP methods that are capable of processing a preformed ADS that has been prepared into its final dosage form while retaining the amorphous nature of the active pharmaceutical ingredient.

### SUMMARY OF THE INVENTION

The present disclosure provides methods of preparing a pharmaceutical composition comprising:
(A) obtaining an amorphous solid dispersion comprising:
   (1) an active pharmaceutical ingredient;
   (2) a pharmaceutically acceptable polymer; and
   (3) an excipient
(B) subjecting the amorphous solid dispersion to an additive manufacturing technique.

In some embodiments, the amorphous solid dispersion is prepared through a mixing process. In some embodiments, the mixing process is either hot melt extrusion or a high energy fusion process. In some embodiments, the amorphous solid dispersion is prepared using a mixing process with a processing speed from about 500 RPM to about 5,000 RPM. In some embodiments, the processing speed is from about 1,000 RPM to about 4,000 RPM. In some embodiments, the processing speed is from about 2,000 RPM to about 3,000 RPM. In some embodiments, the processing speed is about 2,500 RPM. In other embodiments, the amorphous solid dispersion is prepared using a mixing process with a processing speed from about 5 RPM to about 100 RPM. In some embodiments, the processing speed is from about 10 RPM to about 80 RPM. In some embodiments, the processing speed is from about 20 RPM to about 75 RPM.

In some embodiments, the amorphous solid dispersion is prepared through a mixing process with an ejection temperature from about 20° C. to about 250° C. In some embodiments, the ejection temperature is from about 75° C. to about 225° C. In some embodiments, the ejection temperature is from about 100° C. to about 225° C. In some embodiments, the ejection temperature is from about 150° C. to about 200° C. In some embodiments, the ejection temperature is about 160° C.

In some embodiments, the amorphous solid dispersion is prepared through a mixing process with a run time from about 3 seconds to about 5 minutes. In some embodiments, the run time is from about 5 seconds to about 3 minutes. In some embodiments, the run time is from about 10 seconds to about 2 minutes. In some embodiments, the amorphous solid dispersion is prepared through a solvent evaporation process. In some embodiments, the solvent evaporation process is spray drying. In some embodiments, the amorphous solid dispersion is milled. In some embodiments, the amorphous solid dispersion is sieved. In some embodiments, the amorphous solid dispersion is milled and sieved.

In some embodiments, the amorphous solid dispersion is sieved to particle size of less than 1 mm. In some embodiments, the particle size is less than 500 μm. In some embodiments, the particle size is less than 250 μm.

In some embodiments, the additive manufacturing technique is vat photopolymerization, material jetting, binder jetting, powder-bed fusion, material extrusion, directed energy deposition, or sheet lamination. In some embodiments, the additive manufacturing technique is fused deposition modeling, binder spraying, or selective laser sintering. In some embodiments, the additive manufacturing technique comprises exposing the composition to an energy source to form a pattern.

In some embodiments, the additive manufacturing technique comprises exposing the composition to an energy source and the energy source is insufficient to cause the composition to recrystallize. In some embodiments, less than 10% of the composition has recrystallized. In some embodiments, less than 5% of the composition has recrystallized. In some embodiments, less than 2% of the composition has recrystallized. In some embodiments, less than 1% of the composition has recrystallized.

In some embodiments, the energy source has a hatch spacing from about 0.1 μm to about 250 μm. In some embodiments, the hatch spacing is from about 1 μm to about 200 μm. In some embodiments, the hatch spacing is from about 2.5 μm to about 150 μm. In some embodiments, the hatch spacing is about 5 μm, 25 μm, or 100 μm.

In some embodiments, the methods comprise exposing the composition to a laser in a pattern. In some embodiments, the methods comprise depositing a layer of the composition onto a surface in a chamber. In some embodiments, the layer has a layer thickness from about 1 μm to about 5 mm. In some embodiments, the layer thickness is from about 10 μm to about 2.5 mm. In some embodiments, the layer thickness is from about 50 μm to about 1 mm. In some embodiments, the layer thickness is from 50 μm to about 400 μm.

In some embodiments, the layer comprises a surface temperature at its surface different from a chamber temperature in the chamber. In some embodiments, the surface temperature is from about 0° C. to about 250° C. In some embodiments, the surface temperature is from about 50° C. to about 175° C. In some embodiments, the surface temperature is from about 75° C. to about 150° C. In some embodiments, the chamber temperature is from about 25° C. to about 250° C. In some embodiments, the chamber temperature is from about 40° C. to about 200° C. In some embodiments, the chamber temperature is from about 50° C. to about 150° C.

In some embodiments, the pattern is prepared by passing the energy source over the composition with a laser speed from about 0.1 mm/s to about 5,000 mm/s. In some embodiments, the laser speed is from about 0.5 mm/s to about 2,500 mm/s. In some embodiments, the laser speed is from about 1 mm/s to about 2,000 mm/s. In some embodiments, the laser speed is 100 mm/s.

In some embodiments, the energy source is a laser. In some embodiments, the laser comprises a laser power from about 0.1 W to about 250 W. In some embodiments, the laser power is from about 5 mW to about 20 W. In some embodiments, the laser power is from about 50 mW to about 1 W. In some embodiments, the laser power is from about 100 mW to about 500 mW. In some embodiments, the laser comprises a beam size from about 0.25 μm to about 1 mm. In some embodiments, the beam size is from about 1 μm to about 500 μm. In some embodiments, the beam size is from about 2.5 μm to about 100 μm. In some embodiments, the laser has a wavelength from about 50 nm to about 15,000 nm. In some embodiments, the wavelength is from about 5 nm to about 11,000 nm. In some embodiments, the wavelength is from about 200 nm to about 1,000 nm.

In some embodiments, the laser gives the composition an amount of energy equal to an electron laser density from about 2.5 J/mm³ to about 500 J/mm³. In some embodiments, the electron laser density is from about 5 J/mm³ to about 250 J/mm³. In some embodiments, the electron laser density is from about 7.5 J/mm³ to about 50 J/mm³. In some embodiments, the electron laser density is greater than 2.5 J/mm³. In some embodiments, the electron laser density is greater than 5 J/mm³. In some embodiments, the electron laser density is greater than 7.5 J/mm³.

In some embodiments, the amorphous solid dispersion comprises at least 90% of the active pharmaceutical ingredient in the amorphous form. In some embodiments, the amorphous solid dispersion comprises at least 95% of the active pharmaceutical ingredient in the amorphous form. In some embodiments, the pharmaceutical composition comprises at least 99% of the active pharmaceutical ingredient in the amorphous form.

In some embodiments, the active pharmaceutical ingredient and the pharmaceutically acceptable polymer is homogenously mixed together. In some embodiments, the active pharmaceutical ingredient is a poorly soluble drug. In some embodiments, the active pharmaceutical ingredient is a BCS class 2 drug. In some embodiments, the active pharmaceutical ingredient is a BCS class 3 drug. In some embodiments, the active pharmaceutical ingredient is a BCS class 4 drug.

In some embodiments, the active pharmaceutical ingredient is an agent which undergoes degradation at an elevated temperature in a formulation process. In some embodiments, the active pharmaceutical ingredient is chemically sensitive to temperature. In some embodiments, the active pharmaceutical ingredient is chemically sensitive to shear. In some embodiments, the active pharmaceutical ingredient is an agent with a melting point of greater than 60° C. In some embodiments, the melting point is from about 60° C. to about 300° C. In some embodiments, the melting point is from about 80° C. to about 200° C.

In some embodiments, the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives.

In some embodiments, the amorphous solid dispersion comprises from about 1% w/w to about 90% w/w of the active pharmaceutical ingredient. In some embodiments, the amorphous solid dispersion comprises from about 5% w/w to about 50% w/w of the active pharmaceutical ingredient. In some embodiments, the amorphous solid dispersion comprises from about 10% w/w to about 30% w/w of the active pharmaceutical ingredient. In some embodiments, the amorphous solid dispersion n comprises from about 5% w/w to about 30% w/w of the active pharmaceutical ingredient.

In some embodiments, the pharmaceutically acceptable polymer is a cellulosic polymer. In some embodiments, the cellulosic polymer is a neutral cellulosic polymer. In some embodiments, the cellulosic polymer is a charged cellulosic polymer. In some embodiments, the pharmaceutically acceptable polymer is a neutral non-cellulosic polymer. In some embodiments, the neutral non-cellulosic polymer comprises a poly(vinyl acetate), poly(vinylpyrrolidone), poly(ethylene glycol), poly(ethylene oxide), poly(vinyl alcohol), or methacrylate unit. In some embodiments, the pharmaceutically acceptable polymer comprises a poly(vinyl acetate) or a methacrylate unit. In some embodiments, the pharmaceutically acceptable polymer is a poly(vinyl acetate)-co-poly(vinylpyrrolidone) copolymer, dimethylaminoethyl methacrylate-methacrylic acid ester copolymer, ethylacrylate-methylmethacrylate copolymer, poly(vinyl acetate) phthalate, poly(methacrylate ethylacrylate) (1:1) copolymer, poly(methacrylate methylmethacrylate) (1:1) copolymer, poly(methacrylate methylmethacrylate) (1:2) copolymer, or polyvinyl caprolactam-polyvinyl acetate-polyethylene glycol graft copolymer sodium dodecyl sulfate.

In some embodiments, the amorphous solid dispersion comprises from about 5% w/w to about 95% w/w of the pharmaceutically acceptable polymer. In some embodiments, the amorphous solid dispersion comprises from about 50% w/w to about 90% w/w of the pharmaceutically acceptable polymer. In some embodiments, the amorphous solid dispersion comprises from about 60% w/w to about 90% w/w of the pharmaceutically acceptable polymer.

In some embodiments, the excipient is a material that leads to improved energy absorption. In some embodiments, the excipient is a material with a lambda max (kₘₐₓ) equal to the wavelength of the laser. In some embodiments, the lambda max is from about 50 nm to about 15,000 nm. In some embodiments, the lambda max is from about 200 nm to about 11,000 nm. In some embodiments, the lambda max is from about 200 nm to about 1,000 nm. In some embodiments, the excipient is an inorganic material. In some embodiments, the excipient is an aluminum material. In some embodiments, the aluminum material is an aluminum inorganic salt. In some embodiments, the aluminum inorganic salt is bentonite, potassium aluminum silicate, aluminum, aluminum sulfates, sodium aluminum phosphate acidic, sodium aluminum silicate, calcium aluminum silicate, starch aluminum octenyl succinate, or potassium aluminum silicate with a coating of titanium dioxide and/or iron oxide. In some embodiments, the aluminum inorganic salt is potassium aluminum silicate with a coating of titanium dioxide and/or iron oxide. In some embodiments, the inorganic material is iron oxide, titanium oxide, or silicates. In some embodiments, the excipient is an organic material such as a dye. In some embodiments, the dye is carmine, a phthalocyanine, or a diazo compound.

In some embodiments, the amorphous solid dispersion comprises from about 0.01% w/w to about 60% w/w of the excipient. In some embodiments, the amorphous solid dispersion comprises from about 0.1% w/w to about 50% w/w of the excipient. In some embodiments, the amorphous solid dispersion comprises from about 1% w/w to about 30% w/w of the excipient. In some embodiments, the amorphous solid dispersion comprises from about 1% w/w to about 10% w/w of the excipient.

In some embodiments, the amorphous solid dispersion further comprises one or more excipients. In some embodiments, the excipient is a processing aid. In some embodiments, the excipient is an opacifying agent. In other embodiments, the amorphous solid dispersion comprises a flowability excipient. In some embodiments, the flowability excipient is a silicon compound. In some embodiments, the flowability excipient is silicon dioxide. In some embodiments, the amorphous solid dispersion comprises from about 0.1% w/w to about 5% w/w of the flowability excipient. In some embodiments, the amorphous solid dispersion comprises from about 0.5% w/w to about 2.5% w/w of the flowability excipient. In some embodiments, the amorphous solid dispersion comprises from about 0.5% w/w to about 1.5% w/w of the flowability excipient.

In yet another aspect, the present disclosure provides pharmaceutical composition prepared according to the methods described herein.

In another aspect, the present disclosure provides methods of treating or preventing a disease or disorder in a patient comprising administering to the patient in need thereof a therapeutically effective amount of a composition prepared as described herein; wherein the active pharmaceutical ingredient in the composition is sufficient to treat or prevent the disease or disorder.

In still yet another aspect, the present disclosure provides uses of a composition prepared as described herein for the treatment or prevention of a disease or disorder in a patient; wherein the active pharmaceutical ingredient in the composition is sufficient to treat or prevent the disease or disorder.

In another aspect, the present disclosure provides compositions for use in the treatment or prevention of a disease or disorder in a patient comprising a therapeutically effective amount of a composition prepared as described herein; wherein the active pharmaceutical ingredient in the composition is sufficient to treat or prevent the disease or disorder.

Other objects, features, and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows scans from differential scanning calorimetry image of the physical mixture as well as two ASD compositions prepared using either 5 or 25 hatch speed.

FIG. 2 shows the temperature endotherm of the composition.

FIG. 3 shows the pXRD of the physical mixture as well as the printed compositions with either the 5 or 25 hatch speed.

FIG. 4 shows the in vitro dissolution results of a physical mixture relative to an amorphous solid dispersion (ASD) formulated as a capsule relative to the ASD printed using the present methods.

### DESCRIPTION OF ILLUSTRATIVE EMBODIMENTS

In some aspects, the present disclosure relates to methods of using selective laser sintering 3D printing to produce therapeutic drug formulations such as oral formulations such as tablets from pre-formed amorphous formulations. Additionally, these compositions may be used in the treatment or prevention of a disease or disorder that may be treated or prevented by the active pharmaceutical ingredient (API).

The use of SLS and other additive manufacturing techniques to prepare a final dosage form using prepared or processed active pharmaceutical ingredients was not clear as the added energy by the sintering process could have converted the active pharmaceutical ingredient into the crystalline form. Furthermore, these methods maybe used to modify the parameters such that the composition may alter the drug dissolution profile based upon the printing. Additionally, these forms can be prepared with less filler and therefore reducing the pill burden on a patient. Finally, these methods may be used to formulate high drug loading APIs with increased stability and other advantages.

One element of the present methods is a surface temperature of the printing process that has been set about 5 to about 50 degrees below the melting temperature of the polymer and the API depending on the thermal event such as the glass transition temperature or melting temperature responsible for the solubilization or fusion of the drug in the polymer. The thermal event can be determined by using theoretical methods such as thermodynamic Flory-Huggins modeling or by using theoretical solubility parameters. The temperature, either glass transition temperature or melting temperature may also be predicted by using experimental thermal techniques such as differential scanning calorimetry or thermogravimetric analysis. The surface temperature for the methods can be defined as the temperature of the layer exposed to the laser before sintering. The surface temperature can be set and controlled using the heat source placed directly above the print bed. Such heat sources include an infrared heating lamp or an inductive heating source. Surface temperature, as used herein, may be defined as the temperature of the composition. This temperature of the composition for the print layer and represents a threshold temperature that when exposed to a laser source traveling at a specific hatching spacing and speed leads to the formation of amorphous solid dispersions. Using this temperature and adjusted laser parameters, the methods led to the complete amorphous conversion of the physical blend.

In the methods described herein, the methods comprise a chamber temperature during the additive manufacturing process set about 5 to about 50 degrees below the surface temperature. This temperature is also, alternatively, below the glass transition temperature of the polymer in the composition. By way of example, the compositions with Ritonavir had the chamber temperature 15 degrees below the surface temperature. As used herein, the chamber temperature for this disclosure can be defined as a temperature of the build chamber that encases the printing surface. The chamber temperature may be used to aid the temperature increment to the surface temperature but at which no thermal events can occur or be escalated in the physical blend in the reservoir chamber or the print chamber. If the chamber temperature close to the surface temperature or close to any thermal events of any of the components in the physical blend can lead to a print failure due to poor flow of the physical blend, the higher chamber temperature could lead to unwanted melt fusion and agglomeration of the drug and the polymer particles in the reservoir bed and the build chamber. Without wishing to be bound by any theory, the chamber temperature should be controlled with respect to the print time for one layer, for example, the longer the print time the chamber temperature should be set to a temperature further from the thermal event, such as the glass temperature or the melting temperature, or the surface temperature, the chamber temperature should be to prevent print failure. While high chamber temperature can lead to poor flow of the physical blend from the reservoir chamber to the build chamber, prolonged exposure to a high surface temperature can lead to the components to fuse together in the chamber bed; it can also lead to temperature based amorphous conversion of the API instead of laser-based amorphous conversion that has certain disadvantages noted above.

Another element of the process is the hatch spacing or hatch distance (HS), which was set to 25 in the present methods. As used herein, the HS may be defined as the minimum distance between the center of one laser beam to the center of the next laser beam as the laser passes over the chamber to print the pharmaceutical composition and thus may be used to convert the physical blend into an amorphous solid dispersion. For the composition described herein, the compositions that had a hatch spacing more than 25 may leave traces of crystallinity in the produced ASD. This particular parameter was used in the present methods in that it allows the laser to travel across the physical mixture in the print bed in a close-knit pattern which ensures the exposure of the laser to the complete print surface. When the HS was increased, the compositions were observed that the physical blend was not entirely fused and forms a brittle, agglomerated mass of powder which exhibits crystallinity. On the other hand, a low HS along with a low laser speed may be used to maintain high levels of area-related energy densities that results in the formation of ASDs. Without wishing to be bound by any theory, it is believed that the HS is closely related to the laser speed and both these parameters along with the print surface area together determine the print time for each layer where the print time is directly proportional to the surface area and inversely proportional to the HS and the laser speed.

In some embodiments, the laser speed (LS) during the printing process was set within the range of about 25 to about 100 mm/sec. As used herein, the laser speed may be defined as the travel speed of the laser or the exposure time of the laser onto the print surface. This speed should be sufficient for the melt solubilization or melt fusion of the components in the physical blend leading to the formation of amorphous solid dispersion. The lower the laser speed the higher the time required to sinter one layer. During successful printing and complete amorphous conversion, a lower laser speed was used. Furthermore, it was also determined that when the laser speed is reduced the surface temperature should also be reduced as a low laser speed and a high surface temperature leads to a print failure. Without wishing to be bound by any theory, it is believed that prolonged exposure of heat to the surface layer leads to print failure. This particular parameter is useful for obtaining an amorphous composition.

The LS and the HS along with the power of the laser and the thickness of the layer provide the volume related electron laser density. Although this equation provides a good approximation regarding the relationship between the mentioned parameters, it does not take into account several materials associated factors. This equation can provide the density of the laser is exposed over a certain volume but the fraction of the energy absorbed for the melt fusion and solubilization of the physical blend to form an ASD is material specific. In some aspects, the energy input into the system by the laser as the electron laser density may also take into consideration other factors such as surface temperature, chamber temperature, drug load, and formulation components.

$$Electron Laser Density ( \frac{J}{{mm}^{3}} ) = \frac{Laser Power ( w )}{LS \times HS \times LT}$$

Additionally, different energy thresholds are needed to print a tablet as well as simply printing a tablet that is in the amorphous state. The total energy applied to the system is a function of the electron laser density, which is defined by the equation above, and the ability of the composition to absorb a percentage of the energy emitted by the laser. Each composition will have a different electron laser density necessary to overcome each threshold dependent on the composition's capacity to absorb at the wavelength emitted by the laser. Previously, the threshold needed to print an SLS-3DP tablet has been explored but such methods had not been able to reach a threshold to print an SLS-3DP tablet wherein the active pharmaceutical ingredient is in the amorphous form. The most comprehensive report of printing parameters were disclosed within U.S. Patent Application No. 2019/037441. This application contains a list but has no mention of hatch spacing. The list includes the following parameters including a surface temperature 0-200° C. preferably 70-170° C., chamber temperature 25-200° C. preferably 60-150° C., layer thickness 10 mm-0.01 mm, beam size 0.0025-1 mm, scan speed 5 mm/s to 50,000 mm/s preferably 20-300 mm/s, Laser power 0.5 W to 140 W preferably 1.7-8 W, and wavelength 200 nm to 11,000 nm. This patent application describes that the Andrew number for each composition should retain a similar value by modification of either the scan speed or the laser power. This number applied to a composition is believed to influence the release properties of a formulation. It has not been suggested that by combining the electron laser density with a composition's ability to absorb electromagnetic radiation at the emitted wavelength a model can be created the total energy absorbed by the composition to determine the increase in temperature as a result of the laser. Tailoring the surface temperature to the maximum temperature without altering the flow properties enables a successful print in combination with using the minimal energy to overcome the melting point of the drug in composition minimizes the potential degradation that could be induced by the laser. Without wishing to be bound by any theory, it is believed that the combination of the electron laser density, absorption of the composition, hatch spacing and that SLS-3DP does not involve mixing allows the user to design a system that uses the laser energy in combination with surface temperature to create an amorphous 3D-printed tablet.

In some aspects, the present disclosure relates to the preparation of amorphous solid dispersion. While ASDs may be prepared using a variety of different processing methods, not all amorphous solid dispersions are created equal. While this fact may seem counterintuitive, as all amorphous solid dispersions experience solubility enhancement, appear amorphous via characterization techniques, and even ssNMR seems to produce similar domain sizes with different processes. Recently, despite the similarity at a molecular level, differences in ASD performance may be attributed to specific characteristics that are process dependent. For example, the preparation of an ASDs prepared using spray drying, the small particle size produced from the product corresponds to an increased surface area of the particle.

Consequently, the differences in the formulation of the ASDs result in greater drug exposure on the surface, promoting a higher tendency to recrystallize upon storage and rapid drug release for enteric dosage, which is not desired. The discrepancy between amorphous products depends on the amount of drug-exposed on the surface. Stability, SEM (porosity), dissolution, and XPS would be viable tests to differentiate differences between ASD by different processes. These differences come down to how well the API is protected and stabilized by the carrier in which it is processed, the more protected, the lower the tendency to crystalize and release quickly upon dissolution. Therefore, the preparation of ASDs through new methodologies and with new processes is important to developing better and more effective ASDs.

### I. PHARMACEUTICAL COMPOSITIONS

In some aspects, the present disclosure provides pharmaceutical compositions containing an active pharmaceutical ingredient or a pharmaceutically acceptable salt, ester, derivative, analog, pro-drug, or solvates thereof, a pharmaceutically acceptable polymer including polymeric excipients, and electromagnetic energy-absorbing excipient such as an inorganic or organic compound that absorbs electromagnetic energy. These compositions may be amorphous in nature and formulated as an amorphous solid dispersion. In some aspects, the pharmaceutically acceptable polymer and the electromagnetic energy-absorbing excipient may be processed to obtain a compound excipient which is then formulated with the active pharmaceutical ingredient. In some embodiments, these pharmaceutical compositions may be an ASD that is then converted into a final dosage form. In some embodiments, the pharmaceutical composition is substantially, essentially, or entirely free of any other compound.

In some aspects, the present composition may be substantially, essentially, or entirely free from any plasticizer or similar agents which interact with the pharmaceutical composition on the molecular level. Without wishing to be bound by any theory, it is believed that the electromagnetic energy absorbing excipients does not interact with the pharmaceutical composition but rather acts to facilitate the transfer of heat more efficiently.

Additionally, the present compositions are in the amorphous form at a temperature below the melting point of the active pharmaceutical ingredient or below the glass transition temperature of the composition. These present composition may contain some small percentage of crystallinity that is further converted to the amorphous form during the sintering process into the final dosage form. This temperature below the melting point of the active pharmaceutical ingredient or the glass transition temperature of the composition may also be referred to as the thermal event. The temperature at which the composition is converted into the amorphous form or into an amorphous solid dispersion is the surface temperature and maybe at least about 1° C., at least about 5° C., at least about 10° C., at least about 15° C., at least about 20° C., at least about 25° C., at least about 30° C., at least about 35° C., at least about 40° C., or at least about 50° C. below the melting point of the active pharmaceutical ingredient or the glass transition temperature. In some embodiments, the methods used herein comprise using heating the composition to a temperature that is from about 1° C. to about 50° C., from about 5° C. to about 40° C., or from about 10° C. to about 30° C. less than the melting point of the active pharmaceutical ingredient or the glass transition temperature. In some embodiments, the pharmaceutical composition comprises at least 75%, at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.8%, or at least 99.9% of the active pharmaceutical ingredient in the amorphous form.

### A. Active Pharmaceutical Ingredient

The pharmaceutical compositions described herein comprise an active pharmaceutical ingredient. The pharmaceutical compositions described herein contain an active pharmaceutical ingredient in an amount between about 5% to about 95% w/w, between about 10% to about 90% w/w, between about 10% to about 50% w/w, or between about 10% to about 40% w/w of the total composition. In some embodiments, the amount of the active pharmaceutical ingredient is from about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33% 34%, 35%, 36%, 37%, 38%, 39%, 40%, 42%, 44%, 45%, 46%, 48%, 50%, 52%, 54%, 55%, 56%, 58%, 60%, 65%, 70%, 75%, 80%, to about 90% w/w or any range derivable therein. In some embodiments, the pharmaceutical composition is substantially, essentially, or entirely free of any other active pharmaceutical ingredient. In some embodiments, the pharmaceutical compositions may have a ratio of the of the active pharmaceutical ingredient to the electromagnetic energy-absorbing excipient from about 5:1 to about 1:10, from about 2:1 to about 1:5, or from about 1:1 to about 1:3. The ratio may be 5:1, 4:1, 3:1, 2:1, 1.5:1, 1:1, 1:1.5, 1:2, 1:3, 1:4, 1:5, 1:6, 1:8, or 1:10, or any range derivable therein.

In some embodiments, the active pharmaceutical ingredient is classified using the Biopharmaceutical Classification System (BCS), originally developed by G. Amidon, which separates pharmaceuticals for oral administration into four classes depending on their aqueous solubility and their permeability through the intestinal cell layer. According to the BCS, drug substances are classified as follows: Class I-High Permeability, High Solubility; Class II-High Permeability, Low Solubility; Class III-Low Permeability, High Solubility; and Class IV-Low Permeability, Low Solubility.

In particular, typical BCS Class II that may be incorporated into the present pharmaceutical compositions include but are not limited to anti-infectious drugs such as Albendazole, Acyclovir, Azithromycin, Cefdinir, Cefuroxime axetil, Chloroquine, Clarithromycin, Clofazimine, Diloxanide, Efavirenz, Fluconazole, Griseofulvin, Indinavir, Itraconazole, Ketoconazole, Lopinavir, Mebendazole, Nelfinavir, Nevirapine, Niclosamide, Praziquantel, Pyrantel, Pyrimethamine, Quinine, and Ritonavir. Antineoplastic drugs such as Bicalutamide, Cyproterone, Gefitinib, Imatinib, and Tamoxifen. Biologic and Immunologic Agents such as Cyclosporine, Mycophenolate mofetil, Tacrolimus. Cardiovascular Agents such as Acetazolamide, Atorvastatin, Benidipine, Candesartan cilexetil, Carvedilol, Cilostazol, Clopidogrel, Ethylicosapentate, Ezetimibe, Fenofibrate, Irbesartan, Manidipine, Nifedipine, Nilvadipine, Nisoldipine, Simvastatin, Spironolactone, Telmisartan, Ticlopidine, Valsartan, Verapamil, Warfarin. Central Nervous System Agents such as Acetaminophen, Amisulpride, Aripiprazole, Carbamazepine, Celecoxib, Chlorpromazine, Clozapine, Diazepam, Diclofenac, Flurbiprofen, Haloperidol, Ibuprofen, Ketoprofen, Lamotrigine, Levodopa, Lorazepam, Meloxicam, Metaxalone, Methylphenidate, Metoclopramide, Nicergoline, Naproxen, Olanzapine, Oxcarbazepine, Phenytoin, Quetiapine Risperidone, Rofecoxib, and Valproic acid. Dermatological Agents such as Isotretinoin—Endocrine and Metabolic Agents such as Dexamethasone, Danazol, Epalrestat, Gliclazide, Glimepiride, Glipizide, Glyburide (glibenclamide), levothyroxine sodium, Medroxyprogesterone, Pioglitazone, and Raloxifene. Gastrointestinal Agents such as Mosapride, Orlistat, Cisapride, Rebamipide, Sulfasalazine, Teprenone, and Ursodeoxycholic Acid. Respiratory Agents such as Ebastine, Hydroxyzine, Loratadine, and Pranlukast. However, the skilled person will be well aware of other BCS class II drugs which can be used with the pharmaceutical compositions described herein.

Additionally, BCS class III drugs that may be incorporated into the present pharmaceutical compositions include but are not limited to cimetidine, acyclovir, atenolol, ranitidine, abacavir, captopril, chloramphenicol, codeine, colchicine, dapsone, ergotamine, kanamycin, tobramycin, tigecycline, zanamivir, hydralazine, hydrochlorothiazide, levothyroxine, methyldopa, paracetamol, propylthiouracil, pyridostigmine, sodium cloxacillin, thiamine, benzimidazole, didanosine, ethambutol, ethosuximide, folic acid, nicotinamide, nifurtimox, and salbutamol sulfate. However, the skilled person will be well aware of other BCS class III drugs which can be used with the pharmaceutical compositions described herein.

Additionally, BCS class IV drugs that may be incorporated into the present pharmaceutical compositions include but are not limited to hydrochlorothiazide, furosemide, cyclosporin A, itraconazole, indinavir, nelfinavir, ritonavir, saquinavir, nitrofurantoin, albendazole, acetazolamide, azithromycin, senna, azathioprine, chlorthalidone, BI-639667, rifabutin, paclitaxel, curcumin, etoposide, neomycin, methotrexate, atazanavir sulfate, Aprepitant, amphotericin B, amiodarone hydrochloride, or mesalamine. However, the skilled person will be well aware of other BCS class IV drugs which can be used with the pharmaceutical compositions described herein.

While the pharmaceutical compositions and methods described herein can be applied to any BCS class of drugs, BCS class II and IV are of interest for the pharmaceutical compositions described herein. Additionally, other active pharmaceutical ingredients that are of specific consideration are those are those that are high melting point drugs such as a drug that has a melting point of greater than 60° C. Alternatively, the active pharmaceutical ingredients used herein may have a melting point from about 35° C. to about 1,000° C., from about 50° C. to about 750° C., or from about 60° C. to about 200° C. In particular, the melting point may be greater than 25° C., 35° C., 50° C., 60° C., 80° C., 100° C., 125° C., 150° C., 175° C., 200° C., or 250° C.

In some aspects, the present methods may be used to formulate one or more poorly soluble active pharmaceutical ingredients such as deferasirox, etravirine, indomethacin, posaconazole, and ritonavir. Etravirine is a neutral active agent and may be used as a model for other neutral active agents. Deferasirox and indomethacin is a weak acid API and may be used as a model for other weak acid APIs. Posaconazole, itraconazole, and ritonavir are weak base APIs and may be used as models for other weak base APIs.

Suitable active pharmaceutical ingredients may be any poorly water-soluble, biologically active pharmaceutical ingredients or a salt, isomer, ester, ether or other derivative thereof, which include, but are not limited to, anticancer agents, antifungal agents, psychiatric agents such as analgesics, consciousness level-altering agents such as anesthetic agents or hypnotics, nonsteroidal antiinflammatory agents (NSAIDS), anthelminthics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, antiinflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives.

Non-limiting examples of the active pharmaceutical ingredients may include 7-Methoxypteridine, 7-Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone HCl, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol HCl, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine HCl, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil HCl, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin HCl, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cyproheptadine HCl, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem HCl, diloxamide furoate, dimenhydrinate, dimorpholamine, dinitolmide, diosgenin, diphenoxylate HCl, diphenyl, dipyridamole, dirithromycin, disopyramide, disulfiram, diuron, docetaxel, domperidone, donepezil, doxazosin, doxazosin HCl, doxorubicin (neutral), doxorubicin HCl, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine HCl, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine HCl, flunisolide, flunitrazepam, fluocortolone, fluometuron, fluorene, fluorouracil, fluoxetine HCl, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine HCl, haloperidol, hydrochlorothiazide, heptabarbital, heroin, hesperetin, hexachlorobenzene, hexethal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole, itraconazole, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline HCl, mazindol, Meclizine HCl, meclofenamic acid, medazepam, medigoxin, medroxyprogesterone acetate, mefenamic acid, Mefloquine HCl, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin HCl, miconazole, midazolam, mifepristone, miglitol, minocycline, minoxidil, mitomycin C, mitotane, mitoxantrone, mofetilmycophenolate, molindone, montelukast, morphine, Moxifloxacin HCl, nabumetone, nadolol, nalbuphine, nalidixic acid, nandrolone, naphthacene, naphthalene, naproxen, naratriptan HCl, natamycin, nelarabine, nelfinavir, nevirapine, nicardipine HCl, niclosamide, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline HCl, nystatin, oestradiol, ofloxacin, olanzapine, omeprazole, omoconazole, ondansetron HCl, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine HCl, paclitaxel, palifermin, pamidronate, p-aminosalicylic acid, pantoprazole, paramethadione, paroxetine HCl, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine HCl, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin HCl, prednisolone, prednisone, primidone, probarbital, probenecid, probucol, procarbazine, prochlorperazine, progesterone, proguanil HCl, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine HCl, rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole HCl, rosiglitazone, saccharin, salbutamol, salicylamide, salicylic acid, saquinavir, sargramostim, secbutabarbital, secobarbital, sertaconazole, sertindole, sertraline HCl, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin HCl, terbinafine HCl, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine HCl, tibolone, ticlopidine, tinidazole, tioconazole, tirofiban, tizanidine HCl, tolazamide, tolbutamide, tolcapone, topiramate, topotecan, toremifene, tositumomab, tramadol, trastuzumab, trazodone HCl, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine HCl, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, and zopiclone.

In particular aspects, the active pharmaceutical ingredients may be busulfan, taxane, or other anticancer agents; alternatively, itraconazole (Itra) and posaconazole (Posa) or other members of the general class of azole compounds. Exemplary antifungal azoles include a) imidazoles such as miconazole, ketoconazole, clotrimazole, econazole, omoconazole, bifonazole, butoconazole, fenticonazole, isoconazole, oxiconazole, sertaconazole, sulconazole and tioconazole, b) triazoles such as fluconazole, itraconazole, isavuconazole, ravuconazole, Posaconazole, voriconazole, terconazole, and c) thiazoles such as abafungin. Other active pharmaceutical ingredients that may be used with this approach include, but are not limited to, hyperthyroid drugs such as carbimazole, anticancer agents like cytotoxic agents such as epipodophyllotoxin derivatives, taxanes, bleomycin, anthracyclines, as well as platinum compounds and camptothecin analogs. The following active pharmaceutical ingredients may also include other antifungal antibiotics, such as poorly water-soluble echinocandins, polyenes (e.g., Amphotericin B and Natamycin) as well as antibacterial agents (e.g., polymyxin B and colistin), and anti-viral drugs. The active pharmaceutical ingredients may also include a psychiatric agent such as an antipsychotic, anti-depressive agent, or analgesic and/or tranquilizing agents such as benzodiazepines. The active pharmaceutical ingredients may also include a consciousness level-altering agent or an anesthetic agent, such as propofol. The present compositions and the methods of making them may be used to prepare a pharmaceutical composition with the appropriate pharmacokinetic properties for use as therapeutics.

In some aspects, the method may be mostly used with active pharmaceutical ingredients which undergo degradation at an elevated temperature or pressure/shear. The active pharmaceutical ingredients that may be used include those which decompose at a temperature above about 50° C. In some embodiments, the active pharmaceutical ingredients decompose above a temperature of 80° C. In some embodiments, the active pharmaceutical ingredients decompose above a temperature of 100° C. In some embodiments, the active pharmaceutical ingredients decompose above a temperature of 150° C. The active pharmaceutical ingredients that may be used include therein which decompose at a temperature of greater than about 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., or 150° C.

Alternatively, active pharmaceutical ingredients may be one that is sensitive to shear. These active pharmaceutical ingredients are compounds for which the chemical and/or physical properties may change due to friction resulting from the manufacturing process itself, including chemical degradation of a drug or the loss of molecular weight of a polymer as non-limiting examples. The degree of loss of the chemical or physical properties of a compound due to shear is often seen as a function of the degree of mixing (e.g., blade RPM, rotation speed) and the properties of the polymer carrier (e.g. rheological properties).

### B. Excipients

In some aspects, the present disclosure comprises one or more excipients formulated into pharmaceutical compositions including a pharmaceutically acceptable polymer and an electromagnetic energy absorbing excipients. An “excipient” refers to pharmaceutically acceptable carriers that are relatively inert substances used to facilitate administration or delivery of an API into a subject or used to facilitate the processing of an API into drug formulations that can be used pharmaceutically for delivery to the site of action in a subject. Non-limiting examples of excipients include polymer-carriers, stabilizing agents, surfactants, surface modifiers, solubility enhancers, buffers, opacifying agent, encapsulating agents, antioxidants, preservatives, nonionic wetting or clarifying agents, viscosity-increasing agents, and absorption-enhancing agents. In some embodiments, the pharmaceutical composition is substantially, essentially, or entirely free of any other excipient.

### 1. Electromagnetic Energy Absorbing Excipients

In some aspects, the pharmaceutical composition may further comprise one or more inorganic or organic material that promotes the absorbance of electromagnetic energy. In one embodiment, the electromagnetic energy-absorbing excipient is inert and does not interact with the formulation. Without wishing to be bound by any theory, it is believed that the addition of the electromagnetic energy-absorbing excipient increases the ability of the system to readily disperse energy throughout the formulation. By increasing the efficiency of electromagnetic energy when exposed to a laser, it is believed that the addition eliminates the total amount of energy needed to cover the composition into an amorphous form. The addition of these materials thus may be used to create a more favorable formation of an amorphous material such as an amorphous solid dispersion.

In some embodiments, the pharmaceutical compositions of the present disclosure include one or more inorganic and/or organic materials as the electromagnetic energy-absorbing excipient. Some non-limiting examples of electromagnetic energy-absorbing excipient (EEAE) include: Candurin® (potassium aluminum silicate (mica) with a coating of Titanium dioxide and/or iron oxide), Potassium aluminum silicate (PAS), aluminum, aluminum sulfates, sodium aluminum phosphate acidic, sodium aluminum silicate, calcium aluminum silicate, bentonite, starch aluminum octenyl succinate and other aluminum consisting composition. A skilled artisan would be aware of such aluminum based EEAEs which may be used in the pharmaceutical compositions described herein. In some embodiments, the EEAEs may absorb energy at a lambda max from about 50 nm to about 15,000 nm, from about 100 nm to about 11,000 nm, from about 200 nm to about 1,100 nm, or from about 250 nm to about 900 nm. In some embodiments, the energy is from a laser with a lambda max from about 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, 475 nm, 500 nm, 525 nm, 550 nm, 575 nm, 600 nm, 625 nm, 650 nm, 675 nm, 700 nm, 725 nm, 750 nm, 775 nm, 800 nm, 825 nm, 850 nm, 875 nm, 900 nm, 925 nm, 950 nm, 975 nm, 1,000 nm, 1,025 nm, 1,050 nm, 1,075 nm, 1,100 nm, 1,500 nm, 2,000 nm, 2,500 nm, 3,000 nm, 3,500 nm, 4,000 nm, 4,500 nm, 5,000 nm, 5,500 nm, 6,000 nm, 6,500 nm, 7,000 nm, 7,500 nm, 8,000 nm, 8,500 nm, 9,000 nm, 9,500 nm, 10,000 nm, 10,500 nm, 11,000 nm, 12,000 nm, 13,000 nm, 14,000 nm, to about 15,000 nm, or any range derivable therein. Some other non-limiting examples of inorganic electromagnetic energy-absorbing excipients that may be used include iron oxide, titanium oxide, or silicates. In other embodiments, the EEAE may be an organic material, such as a dye. Some non-limiting examples of dyes which may be used include carmine, phthalocyanine, and diazos.

The EEAE may further comprise a material that is free flowing and comprises small partiles. These particles may be less than 100 μm, less than 75 μm, or less than 50 μm. In particular, the EEAE may further comprise a density from about 1.0 g/cm³ to about 6.0 g/cm³, from about 2.0 g/cm³ to about 5.0 g/cm³, or from about 2.5 g/cm³ to about 3.5 g/cm³. The density of the EEAE may be from about 0.5 g/cm³, 1.0 g/cm³, 1.5 g/cm³, 2.0 g/cm³, 2.5 g/cm³, 3.0 g/cm³, 3.5 g/cm³, 4.0 g/cm³, 4.5 g/cm³, 5.0 g/cm³, 5.5 g/cm³, 6.0 g/cm³, to about 7.5 g/cm³, or any range derivable therein.

In some embodiments, the EEAE is a compound or composition that is already an FDA approved excipient for human consumption. One example of an EEAE that is approved for human consumption and may be incorporated within the pharmaceutical composition is Candurin®. Candurin® is not soluble in water or other biorelevant conditions making it not be completely digested upon consumption but rather only subject to extraction by stomach acids. Candurin® and other aluminum derivatives are often used as a commercially available food additive in confections, candy, decorations, and beverages at maximum concentrations of 1.25%, equating to a range of 10 mg/kg-323 mg/kg/day. Candurin® contains pearlescent pigments achieve their different coloring effects by using different degrees of titanium oxide and/or iron oxide around a potassium aluminum silicate (PAS) core. The pearlescent color effect results from the partial transmittance and partial reflection of light as well as interference of light through the platelets. PAS-BPP comes in three types all types (types I-III) and may be used in this application. In particular, it is noted that PAS-BPP is expected to have excellent thermal stability during food processing and storage, as the thermal conditions experienced are mild in comparison to which the PAS-BPP is made (900 degree Celsius). Therefore, any Candurin® may be used in this application.

Furthermore, the pharmaceutical composition described herein have a concentration of the electromagnetic energy-absorbing excipient ranging from about 0.01% to about 80% w/w. In some embodiments, the amount of electromagnetic energy-absorbing excipient is from about 0.1% to about 60% w/w, from about 0.5% to about 50% w/w, 1% to about 40% w/w, 1% to about 15% w/w, or 2% to about 10% w/w, wherein the weight is measured against the entire composition weight. The amount of electromagnetic energy-absorbing excipient may be from about 1%, 2%, 3%, 4%, 5%, 6%, 7%, 8%, 9%, 10%, 15%, 20%, 25%, 30%, 40%, 50%, 60%, 70%, to about 80%, or any range derivable therein. In some embodiments, the pharmaceutical composition is substantially, essentially, or entirely free of any other electromagnetic energy-absorbing excipient.

### 2. Pharmaceutically Acceptable Polymers

In some aspects, the present disclosure provides compositions which may further comprise a pharmaceutically acceptable polymer. In some embodiments, the polymer (polymer carrier) has been approved for use in a pharmaceutical formulation and is known to undergo softening or increased pliability when raised above a specific temperature without substantially degrading.

When a pharmaceutically acceptable polymer is present in the composition, the pharmaceutically acceptable polymer is present in the composition at a level between 1% to 90% w/w, between 10% to 80% w/w, between 20% to 70% w/w, between 30% to 70% w/w, between 40% to 60% w/w. In some embodiments, the amount of the pharmaceutically acceptable polymer is from about 5%, 10%, 15%, 50%, 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, to about 90% w/w or any range derivable therein. In some embodiments, the pharmaceutical composition is substantially, essentially, or entirely free of any other pharmaceutically acceptable polymer.

Within the compositions described herein, a single polymer or a combination of multiple polymers may be used. In some embodiments, the polymers used herein may fall within two classes: cellulosic and non-cellulosic. These classes may be further defined by their respective charge into neutral and ionizable. Ionizable polymers have been functionalized with one or more groups which are charged at a physiologically relevant pH. Some non-limiting examples of neutral non-cellulosic polymers include polyvinyl pyrrolidone, polyvinyl alcohol, copovidone, and poloxamer. Within this class, in some embodiments, pyrrolidone containing polymers are particularly useful. Some non-limiting examples of charged cellulosic polymers include cellulose acetate phthalate and hydroxypropyl methylcellulose acetate succinate. Finally, some non-limiting examples of neutral cellulosic polymers include hydroxypropyl cellulose, hydroxypropyl methylcellulose, hydroxyethylcellulose, and hydroxymethyl cellulose.

Some specific pharmaceutically acceptable polymers which may be used include, for example, Eudragit™ RS PO, Eudragit™ S100, Kollidon SR (poly(vinyl acetate)-co-poly(vinylpyrrolidone) copolymer), Ethocel™ (ethylcellulose), HPC (hydroxypropylcellulose), cellulose acetate butyrate, poly(vinylpyrrolidone) (PVP), poly(ethylene glycol) (PEG), poly(ethylene oxide) (PEO), poly(vinyl alcohol) (PVA), hydroxypropyl methylcellulose (HPMC), ethylcellulose (EC), hydroxyethylcellulose (HEC), carboxymethyl cellulose and alkali metal salts thereof, such as sodium salts sodium carboxymethyl-cellulose (CMC), dimethylaminoethyl methacrylate—methacrylic acid ester copolymer, carboxymethylethyl cellulose, carboxymethyl cellulose butyrate, carboxymethyl cellulose propionate, carboxymethyl cellulose acetate butyrate, carboxymethyl cellulose acetate propionateethylacrylate—methylmethacrylate copolymer (GA-MMA), C-5 or 60 SH-50 (Shin-Etsu Chemical Corp.), cellulose acetate phthalate (CAP), cellulose acetate trimelletate (CAT), poly(vinyl acetate) phthalate (PVAP), hydroxypropylmethylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, poly(methacylic acid-co-methyl methacrylate 1:2), poly(methacrylic acid-co-methyl methacrylate 1:1), Poly(methyl acrylate-co-methyl methacrylate-co-methacrylic acid 7:3:1), poly(butyl methacrylate-co-(2-dimethylaminoethyl) methacrylate-co-methyl methacrylate 1:2:1), poly(ethyl acrylate-co-methyl methacrylate 2:1), poly(ethyl acrylate-co-methyl methacrylate 2:1), poly(ethyl acrylate-co-methyl methacrylate-co-trimethylammonioethyl methacrylate chloride 1:2:0.2), poly(ethyl acrylate-co-methyl methacrylate-co-trimethylammonioethyl methacrylate chloride 1:2:0.1), Eudragit L-30-D™ (MA-EA, 1:1), Eudragit L-100-55™ (MA-EA, 1:1), hydroxypropylmethylcellulose acetate succinate (HPMCAS), polyvinyl caprolactam-polyvinyl acetate-PEG graft copolymer, polyvinyl alcohol/acrylic acid/methyl methacrylate copolymer, polyalkylene oxide, Coateric™ (PVAP), Aquateric™ (CAP), and AQUACOAT™ (HPMCAS), polycaprolactone, starches, pectins, chitosan or chitin and copolymers and mixtures thereof, and polysaccharides such as tragacanth, gum arabic, guar gum, and xanthan gum.

Additional pharmaceutically acceptable polymers that may be used in the presently disclosed pharmaceutical compositions include but are not limited to polyethylene oxide; polypropylene oxide; polyvinylpyrrolidone; polyvinylpyrrolidone-co-vinyl acetate; acrylate and methacrylate copolymers; polyethylene; polycaprolactone; polyethylene-co-polypropylene; alkyl celluloses such as methylcellulose; hydroxyalkyl celluloses such as hydroxymethyl cellulose, hydroxyethylcellulose, hydroxypropyl cellulose, and hydroxy butyl cellulose; hydroxyalkyl alkyl celluloses such as hydroxyethyl methylcellulose and hydroxypropyl methylcellulose; starches, pectins; polysaccharides such as tragacanth, gum arabic, guar gum, and xanthan gum. One embodiment of the pharmaceutically acceptable polymer is poly(ethylene oxide) (PEO), which can be purchased commercially from companies such as the Dow Chemical Company, which markets PEO under the POLY OX® exemplary grades of which can include WSR N80 having an average molecular weight of about 200,000; 1,000,000; and 2,000,000.

### 3. Other Excipients

In some aspects, the present disclosure provides pharmaceutical compositions that may further comprise one or more additional excipients. The excipients (also called adjuvants) that may be used in the presently disclosed compositions and composites, while potentially having some activity in their own right, for example, antioxidants, are generally defined for this application as compounds that enhance the efficiency and/or efficacy of the active pharmaceutical ingredient. It is also possible to have more than one active agent in a given solution so that the particles formed contain more than one active agent. In particular, the compositions may further comprise one or more flowability excipients such as a silicon compound. The silicon compound may include an oxide of silicon such as silicon dioxide.

Any pharmaceutically acceptable excipient known to those of skill in the art may be used to produce the pharmaceutical compositions disclosed herein. Examples of excipients for use with the present disclosure include, lactose, glucose, starch, calcium carbonate, kaolin, crystalline cellulose, silicic acid, water, simple syrup, glucose solution, starch solution, gelatin solution, carboxymethyl cellulose, shellac, methyl cellulose, polyvinyl pyrrolidone, dried starch, sodium alginate, powdered agar, calcium carmelose, a mixture of starch and lactose, sucrose, butter, hydrogenated oil, a mixture of a quaternary ammonium base and sodium lauryl sulfate, glycerine and starch, lactose, bentonite, colloidal silicic acid, talc, stearates, and polyethylene glycol, sorbitan esters, polyoxyethylene sorbitan fatty acid esters, polyoxyethylene alkyl ethers, poloxamers (polyethylene-polypropylene glycol block copolymers), sucrose esters, sodium lauryl sulfate, oleic acid, lauric acid, vitamin E TPGS, polyoxyethylated glycolysed glycerides, dipalmitoyl phosphadityl choline, glycolic acid and salts, deoxycholic acid and salts, sodium fusidate, cyclodextrins, polyethylene glycols, polyglycolyzed glycerides, polyvinyl alcohols, polyacrylates, polymethacrylates, polyvinylpyrrolidones, phosphatidyl choline derivatives, cellulose derivatives, biocompatible polymers selected from poly(lactides), poly(glycolides), poly(lactide-co-glycolides), poly(lactic acid)s, poly(glycolic acid)s, poly(lactic acid-co-glycolic acid)s and blends, combinations, and copolymers thereof.

As stated, excipients and adjuvants may be used in the pharmaceutical composition to enhance the efficacy and efficiency of the active agent in the pharmaceutical composition. Additional non-limiting examples of compounds that can be included are binders, carriers, cryoprotectants, lyoprotectants, surfactants, fillers, stabilizers, polymers, protease inhibitors, antioxidants, bioavailability enhancers, and absorption enhancers. The excipients may be chosen to modify the intended function of the active ingredient by improving flow, or bioavailability, or to control or delay the release of the API. Specific nonlimiting examples include: sucrose, trehalose, Span 80, Span 20, Tween 80, Brij 35, Brij 98, Pluronic, sucroester 7, sucroester 11, sucroester 15, sodium lauryl sulfate (SLS, sodium dodecyl sulfate. SDS), dioctyl sodium sulphosuccinate (DSS, DOSS, dioctyl docusate sodium), oleic acid, laureth-9, laureth-8, lauric acid, vitamin E TPGS, Cremophor® EL, Cremophor® RH, Gelucire® 50/13, Gelucire® 53/10, Gelucire® 44/14, Labrafil®, Solutol® HS, dipalmitoyl phosphatidyl choline, glycolic acid and salts, deoxycholic acid and salts, sodium fusidate, cyclodextrins, polyethylene glycols, Labrasol®, polyvinyl alcohols, polyvinyl pyrrolidones, and tyloxapol. In particular, the composition may further comprise one or more silicon compounds such as silicon dioxide that improves the flowability of the composition.

The stabilizing carrier may also contain various functional excipients, such as: hydrophilic polymer, antioxidant, super-disintegrant, surfactant including amphiphilic molecules, wetting agent, stabilizing agent, retardant, similar functional excipient, or a combination thereof, and plasticizers including citrate esters, polyethylene glycols, PG, triacetin, diethyl phthalate, castor oil, and others known to those of ordinary skill in the art. Extruded material may also include an acidifying agent, adsorbent, alkalizing agent, buffering agent, colorant, flavorant, sweetening agent, diluent, opaquing, complexing agent, fragrance, preservative or a combination thereof.

Compositions with enhanced solubility may comprise a mixture of the active pharmaceutical ingredient and an additive that enhances the solubility of the active pharmaceutical ingredient. Examples of such additives include but are not limited to surfactants, polymer-carriers, pharmaceutical carriers, thermal binders, or other excipients. A particular example may be a mixture of the active pharmaceutical ingredient with a surfactant or surfactant, the active pharmaceutical ingredient with a polymer or polymers, or the active pharmaceutical ingredient with a combination of a surfactant and polymer carrier or surfactants and polymer-carriers. A further example is a composition where the active pharmaceutical ingredient is a derivative or analog thereof.

In some embodiments, the pharmaceutical compositions may further comprise one or more surfactants. Surfactants that can be used in the disclosed pharmaceutical compositions to enhance solubility include those known to a person of ordinary skill. Some particular non-limiting examples of such surfactants include but are not limited to sodium dodecyl sulfate, dioctyl docusate sodium, Tween 80, Span 20, Cremophor® EL or Vitamin E TPGS.

Solubility can be indicated by peak solubility, which is the highest concentration reached of a species of interest over time during a solubility experiment conducted in a specified medium at a given temperature. The enhanced solubility can be represented as the ratio of peak solubility of the agent in a pharmaceutical composition of the present disclosure compared to peak solubility of the reference standard agent under the same conditions. Preferably, an aqueous buffer with a pH in the range of from about pH 4 to pH 8, about pH 5 to pH 8, about pH 6 to pH 7, about pH 6 to pH 8, or about pH 7 to pH 8, such as, for example, pH 4.0, 4.5, 5.0, 5.5, 6.0, 6.2, 6.4, 6.6, 6.7, 6.8, 6.9, 7.0, 7.1, 7.2, 7.4, 7.6, 7.8, or 8.0, may be used for determining peak solubility. This peak solubility ratio can be about 2:1, 3:1, 4:1, 5:1, 6:1, 7:1, 8:1, 9:1, 10:1, 12:1, 15:1, 20:1, 25:1, 30:1, 35:1, 40:1, 45:1, 50:1, 55:1 or higher.

Compositions of the active pharmaceutical ingredient that enhance bioavailability may comprise a mixture of the active pharmaceutical ingredient and one or more pharmaceutically acceptable adjuvants that enhance the bioavailability of the active pharmaceutical ingredient. Examples of such adjuvants include but are not limited to enzyme inhibitors. Particular examples are such enzyme inhibitors include but are not limited to inhibitors that inhibit cytochrome P-450 enzyme and inhibitors that inhibit monoamine oxidase enzyme. Bioavailability can be indicated by the Cₘₐₓ or the AUC of the active pharmaceutical ingredient as determined during in vivo testing, where Cₘₐₓ is the highest reached blood level concentration of the active pharmaceutical ingredient over time of monitoring and AUC is the area under the plasma-time curve. Enhanced bioavailability can be represented as the ratio of Cₘₐₓ or the AUC of the active pharmaceutical ingredient in a pharmaceutical composition of the present disclosure compared to Cₘₐₓ or the AUC of the reference standard the active pharmaceutical ingredient under the same conditions. This Cₘₐₓ or AUC ratio reflecting enhanced bioavailability can be about 5:1, 6:1, 7:1, 8:1, 9:1, 10:1, 12:1, 15:1, 20:1, 25:1, 30:1, 35:1, 40:1, 45:1, 50:1, 55:1, 60:1, 65:1, 70:1, 75:1, 80:1, 85:1, 90:1, 95:1, 98:1, 99:1, 100:1 or higher.

In other aspects, the present compositions may further comprise one or more opacifying agents which modulate the amount of energy absorbed by the composition. Opacifying agents include such compounds as titanium oxide and alter the clarity and ability of electromagnetic energy to be absorbed by the compositions. Alternatively, these compositions may alter the amount of energy needed to achieve appropriate processing of the compositions. Some non-limiting examples of opacifying agents include those taught by U.S. Pat. Nos. 4,009,139, 5,571,334, and PCT Patent Application No. WO 2020/122950, the entire contents of which are hereby incorporated by reference. Some non-limiting examples of opacifying agents including Aerosil®, Cab-O Si®, or other silicon dioxides, aluminum hydroxide, alumina, aluminum silicate, arachidic acid, barium sulfate, bentonite, calamine, calcium carbonate, calcium phosphate dibasic, calcium phosphate tribasic, calcium silicate, calcium sulfate, ceric oxide, cetyl alcohol, activated charcoal, charcoal, diatomaceous earth, erucamide, ethylene glycol monosterate, Fuller's earth, guanine, hectorite, kaolin, magnesium aluminum silicate, magnesium carbonate, magnesium oxide, magnesium phosphate tribasic, magnesium silicate, magnesium trisilicate, myristic acid, palmitic acid, silica, stannic oxide, stearic acid amide, stearoyl monoethanolamine sterate, stearyl palmitate, talc, titanium dioxide, Veegum® or other granular magnesium aluminum silicates, zinc carbonate basic, zirconium oxide, or zirconium silicate.

In some aspects, the amount of the excipient in the pharmaceutical composition is from about 0.1% to about 20% w/w, from about 0.25% to about 10% w/w, from about 0.5% to about 7.5% w/w, or from about 0.5% to about 5% w/w. The amount of the excipient in the pharmaceutical composition comprises from about 0.1%, 0.2%, 0.25%, 0.3%, 0.4%, 0.5%, 0.6%, 0.7%, 0.75%, 0.8%, 0.9%, 1%, 1.25%, 1.5%, 1.5%, 1.75%, 2%, 2.5%, 3%, 3.5%, 4%, 4.5%, 5%, 5.5%, 6%, 6.5%, 7%, 7.5%, 8%, 9%, to about 10% w/w, or any range derivable therein, of the total pharmaceutical composition. In one embodiment, the amount of the excipient in the pharmaceutical composition is at 0.25% to 2.5% w/w of the total weight of the pharmaceutical composition.

### II. ADDITIVE MANUFACTURING METHODS

In some aspects, the pharmaceutical compositions described herein are processed in a final dosage form. The granules that are produced by the process may be further processed into a capsule or a tablet. Before formulation into a capsule or tablet, the granule may be further milled before being compressed into the capsule or tablet.

In other aspects, the pharmaceutical compositions described herein may also be used in an additive manufacturing platform. Some of the additive manufacturing platforms that may be used herein include 3D printing such as selective laser sintering or selective laser melting. Alternatively, a method such as stereolithography or fused deposition modeling may be used to obtain the final pharmaceutical composition.

These pharmaceutical compositions may be processed through laser sintering wherein a laser is aimed at a specific point on the pharmaceutical composition such that material is bound together to create a solid form. The laser is passed over the surface in a sufficient amount of time and sufficient location to produce the desired dosage form. The method relates to the use of the laser-based upon the power of the laser such as the peak laser power rather than the laser duration. The method often will make use of a pulsed laser. The laser used in these methods often is a high power laser such as a carbon dioxide laser. The process builds up the dosage form using cross-sections of the material through multiple scanning passes over the material. Additionally, the chamber of the 3D printer device may also be preheated to a temperature just below the melting point of the pharmaceutical composition such as the melting point of the composition as a whole or the active pharmaceutical ingredient, the pharmaceutically acceptable polymer, or the combination. Furthermore, the method may be used without the need for a secondary feeder of material into the chamber of the device.

In some embodiments, the additive manufacturing techniques used in the present methods may include selective laser sintering 3D printing. This method may comprise use of a laser onto a composition that has been deposited into a chamber at particular locations. The laser acts to sinter the composition into an amorphous form that may be used as a pharmaceutical composition. The formation of the final product is based upon the energy of the laser as well as the properties of the composition and the temperature of the composition and the chamber that the compositions are deposited into.

In the first part of the selective laser sintering process, the composition is deposited onto a surface in the chamber. The deposition of the composition may result in a layer, wherein the layer of the composition has a layer thickness (LT) from about 0.1 μm to about 100 mm, from about 1 μm to about 100 mm, from about 10 μm to about 100 mm, from about 50 μm to about 10 mm, from about 50 μm to about 1 mm, or from about 50 μm to about 100 μm. The layer thickness may be from about 0.1 μm, 1 μm, 10 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 175 μm, 200 μm, 250 μm, 300 μm, 350 μm, 400 μm, 450 μm, 500 μm, 600 μm, 700 μm, 750 μm, 800 μm, 900 μm, 1 mm, 5 mm, 10 mm, 25 mm, 50 mm, 75 mm, to about 100 mm.

The composition deposited into the surface in the chamber may be heated to a temperature, known as the surface temperature. This surface temperature may be used to provide additional energy to the composition to assist the conversion of the active pharmaceutical ingredient. The surface temperature may be a temperature form about 0° C. to about 500° C., from about 0° C. to about 250° C., from about 25° C. to about 250° C., from about 50° C. to about 175° C., or from about 75° C. to about 150° C. The surface temperature may be a temperature from about 0° C., 25° C., 50° C., 60° C., 70° C., 75° C., 80° C., 90° C., 100° C., 110° C., 120° C., 125° C., 130° C., 140° C., 150° C., 160° C., 170° C., 180° C., 190° C., 200° C., 210° C., 220° C., 230° C., 240° C., 250° C., 275° C., 300° C., 350° C., 400° C., 450° C., to about 500° C., or any range derivable.

Furthermore, the chamber may also be heated to a temperature known as the chamber temperature. The chamber temperature may be a temperature form about 0° C. to about 500° C., from about 0° C. to about 250° C., from about 25° C. to about 250° C., from about 50° C. to about 175° C., or from about 75° C. to about 150° C. The surface temperature may be a temperature from about 0° C., 25° C., 50° C., 60° C., 70° C., 75° C., 80° C., 90° C., 100° C., 110° C., 120° C., 125° C., 130° C., 140° C., 150° C., 160° C., 170° C., 180° C., 190° C., 200° C., 210° C., 220° C., 230° C., 240° C., 250° C., 275° C., 300° C., 350° C., 400° C., 450° C., to about 500° C., or any range derivable. In some embodiments, the chamber temperature is at least 1° C., at least 5° C., at least 10° C., at least 15° C., at least 20° C., at least 25° C., or at least 50° C. less than the surface temperature. The chamber temperature may be from 1° C. to about 50° C., 5° C. to about 25° C., 10° C. to about 25° C., or 10° C. to about 20° C. less than the surface temperature.

Once the composition has been deposited therein, the composition is exposed to a laser to sinter the composition to obtain the final pharmaceutical composition. The parameters of the laser may be used in obtaining an amorphous composition from the composition deposited in the chamber. The particular laser used by the process may further comprise a laser power from about 0.1 W to about 250 W, from about 0.5 W to about 150 W, from about 1 W to about 100 W, or from about 1 W to about 10 W. The laser used herein may have a laser power from about 0.1 W, 0.5 W, 1 W, 2 W, 3 W, 4 W, 5 W, 6 W, 7 W, 8 W, 9 W, 10 W, 15 W, 20 W, 25 W, 30 W, 35 W, 40 W, 45 W, 50 W, 60 W, 70 W, 80 W, 90 W, 100 W, 125 W, 150 W, 200 W, to about 250 W, or any range derivable therein. The particular laser used may include a high power laser such as carbon dioxide laser, lamp or diode, pumped ND:YAG laser, and disk or fiber lasers. In some embodiment, a 2.3 watt solid diode 455 nm wavelength (visible light, bright blue) laser may be used. The laser used may emit light with a wavelength from about 50 nm to about 15,000 nm, from about 200 nm to about 11,000 nm, or from about 200 nm to about 1,000 nm. The wavelength may be 50 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, 475 nm, 500 nm, 525 nm, 550 nm, 575 nm, 600 nm, 625 nm, 650 nm, 675 nm, 700 nm, 725 nm, 750 nm, 775 nm, 800 nm, 825 nm, 850 nm, 875 nm, 900 nm, 925 nm, 950 nm, 975 nm, 1,000 nm, 1,025 nm, 1,050 nm, 1,075 nm, 1,100 nm, 1,500 nm, 2,000 nm, 2,500 nm, 3,000 nm, 3,500 nm, 4,000 nm, 4,500 nm, 5,000 nm, 5,500 nm, 6,000 nm, 6,500 nm, 7,000 nm, 7,500 nm, 8,000 nm, 8,500 nm, 9,000 nm, 9,500 nm, 10,000 nm, 10,500 nm, 11,000 nm, 12,000 nm, 13,000 nm, 14,000 nm, to about 15,000 nm, or any range derivable therein. Furthermore, the laser used may have a specific beam size that indicates the size of the laser that strikes any particular point of the composition at a given time. The methods may further comprise using a laser with a beam size from about 0.1 μm to about 10 mm, from about 0.25 μm to about 1 mm, from about 1 μm to about 500 μm, or from about 2.5 μm to about 100 μm. The beam size may be a size from about 0.1 μm, 0.5 μm, 1 μm, 2.5 μm, 5 μm, 7.5 μm, 10 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 250 μm, 500 μm, 750 μm, 1 mm, to about 5 mm, or any range derivable therein.

The laser may be used to sinter the composition in a pattern. During the sintering process, the laser traces a pattern over the composition to prepare the final pharmaceutical composition. The pattern is prepared by passing the laser over the composition at a specific speed known as the laser speed (LS). The laser speed may be from about 0.1 mm/s to about 100,000 mm/s, from about 0.5 mm/s to about 50,000 mm/s, from about 10 mm/s to about 1,000 mm/s, or from about 25 mm/s to about 250 mm/s. The laser speed may be from about 0.1 mm/s, 0.25 mm/s, 0.5 mm/s, 1 mm/s, 5 mm/s, 10 mm/s 15 mm/s, 20 mm/s, 25 mm/s, 30 mm/s, 35 mm/s, 40 mm/s, 45 mm/s, 50 mm/s, 55 mm/s, 60 mm/s, 65 mm/s, 70 mm/s, 75 mm/s, 80 mm/s, 85 mm/s, 90 mm/s, 95 mm/s, 100 mm/s, 105 mm/s, 110 mm/s, 115 mm/s, 120 mm/s, 125 mm/s, 150 mm/s, 200 mm/s, 250 mm/s, 500 mm/s, 1,000 mm/s, 5,000 mm/s, 25,000 mm/s, 50,000 mm/s, to about 100,000 mm/s, or any range derivable therein. Furthermore, the laser may pass in a pattern over the composition in the surface of the chamber. The distances between the lines in the laser's pass are known as hatches. The distance between each successive laser pass is known as the hatch spacing. The methods used herein may include using a hatch spacing from about 0.1 μm to about 250 μm, from about 5 μm to about 100 μm, from about 10 μm to about 75 μm, from about 10 μm to about 50 μm, or to about 10 μm to about 40 μm. The hatch spacing may be from about 0.1 μm, 0.5 μm, 1 μm, 1 μm, 5 μm, 10 μm, 15 μm, 17.5 μm, 20 μm, 21 μm, 22 μm, 22.5 μm, 23 μm, 24 μm, 25 μm, 26 μm, 27 μm, 27.5 μm, 28 μm, 29 μm, 30 μm, 32.5 μm, 35 μm, 37.5 μm, 40 μm, 45 μm, 50 μm, 60 μm, 70 μm, 80 μm, 90 μm, to about 100 μm, or any range derivable therein.

Finally, the combination of the chamber temperature and the surface temperature may be used to combine with the laser energy to provide sufficient energy to obtain an amorphous solid dispersion based dosage form. The amount of energy that the laser imparts into the pharmaceutical composition is calculated as the electron laser density. Electron laser density (i.e., fluence) may be calculated using the following formula:

$$Electron Laser Density ( \frac{J}{{mm}^{3}} ) = \frac{Laser Power ( w )}{LS \times HS \times LT}$$

The electron laser density may be an amount of energy imparted from the laser from about 1 J/mm³ to about 500 J/mm³, from about 2.5 J/mm³ to about 500 J/mm³, from about 5 J/mm³ to about 250 J/mm³, from about 7.5 J/mm³ to about 100 J/mm³, or from about 7.5 J/mm³ to about 50 J/mm³. The electron laser density is from about 1 J/mm³, 1.5 J/mm³, 2 J/mm³, 2.5 J/mm³, 3 J/mm³, 3.5 J/mm³, 4 J/mm³, 4.5 J/mm³, 5 J/mm³, 5.5 J/mm³, 6 J/mm³, 6.5 J/mm³, 7 J/mm³, 7.5 J/mm³, 8 J/mm³, 8.5 J/mm³, 9 J/mm³, 9.5 J/mm³, 10 J/mm³, 12.5 J/mm³, 15 J/mm³, 17.5 J/mm³, 20 J/mm³, 25 J/mm³, 50 J/mm³, 75 J/mm³, 100 J/mm³, 150 J/mm³, 200 J/mm³, 250 J/mm³, 300 J/mm³, 400 J/mm³, to about 500 J/mm³, or any range derivable therein.

### III. MANUFACTURING METHODS

### A. Hot Melt Extrusion and Thermokinetic Mixing Process

Thus, in one aspect, the present disclosure provides pharmaceutical compositions which may be prepared using a thermal or fusion-based high energy process. Such process may include hot melt extrusion, hot melt granulation, melt mixing, spray congealing, sintering/curing, injection molding, or a thermokinetic mixing process such as the KinetiSol method. Similar thermal processing methods are described in LaFountaine et al., 2016a, Keen et al., 2013, Vynckier et al., 2014, Lang et al., 2014, Repka et al., 2007, Crowley et al., 2007, DiNunzio et al., 2010a, DiNunzio et al., 2010b, DiNunzio et al., 2010c, DiNunzio et al., 2010d, Hughey et al., 2010, Hughey et al., 2011, LaFountaine et al., 2016b, and Prasad et al., 2016, all of which are incorporated herein by reference. In some embodiments of these present disclosure, the pharmaceutical compositions may be prepared using a thermal process such as hot melt extrusion or hot melt granulation. In other embodiments, a fusion based process including thermokinetic mixing process such as those described at least in U.S. Pat. Nos. 8,486,423 and 9,339,440, the entire contents of which are herein incorporated by reference.

A non-limiting list of instruments which may be used to thermally process the pharmaceutical compositions described herein include hot melt extruders available from ThermoFisher®, such as a minilab compounder, or Leistritz®, such as a twin-screw extruder. Alternatively, a fusion-based high energy process instrument that does not require external heat input, including such as a thermokinetic mixer as described in U.S. Pat. Nos. 8,486,423 and 9,339,440 may be used to process the pharmaceutical composition.

In some aspects, the mixing process may comprise heating the composition to an ejection temperature from about 20° C. to about 300° C. In some embodiments, the ejection temperature is from about 150° C. to about 250° C., from about 75° C. to about 225° C., from about 100° C. to about 225° C., or from about 150° C. to about 200° C. The ejection temperature that may be used is from about 60° C., 65° C., 70° C., 75° C., 80° C., 90° C., 92° C., 94° C., 96° C., 98° C., 100° C., 102° C., 104° C., 106° C., 108° C., 110° C., 112° C., 114° C., 116° C., 118° C., 120° C., 125° C., 130° C., 135° C., 140° C., 145° C., 150° C., 155° C., 160° C., 165° C., 170° C., 175° C., 180° C., 190° C., 200° C., 210° C., 220° C., 225° C., 230° C., 240° C., 250° C., 260° C., 270° C., 280° C., 290° C., to about 300° C. or any range derivable therein.

In some embodiments, the mixing process comprises mixing the components into forming the amorphous solid dispersion at a processing speed less than 5000 rpm, less than 4,000 rpm, less than 3,000 rpm, or less than 100 rpm, less than 80 rpm, or less than 75 rpm. The processing speed may be from from about 2 rpm, 3 rpm, 4 rpm, 5 rpm, 10 rpm, 15 rpm, 20 rpm, 25 rpm, 30 rpm, 35 rpm, 40 rpm, 45 rpm, 50 rpm, 55 rpm, 60 rpm, 65 rpm, 70 rpm, 75 rpm, 80 rpm, 85 rpm, 90 rpm, 95 rpm, to about 100 rpm, or any range derivable therein. The processing speed may be from from about 200 rpm, 300 rpm, 400 rpm, 500 rpm, 1,000 rpm, 1,500 rpm, 2,000 rpm, 2,500 rpm, 3,000 rpm, 3,500 rpm, 4,000 rpm, 4,500 rpm, 5,000 rpm, 5,500 rpm, 6,000 rpm, 6,500 rpm, 7,000 rpm, 7,500 rpm, 8,000 rpm, 8,500 rpm, 9,000 rpm, 9,500 rpm, to about 10,000 rpm, or any range derivable therein.

The extrudate produced following the extrusion process will generally comprise the active agent and the pharmaceutically acceptable polymer. The extrudate may be in the form of granules of a desired mesh size or diameter, rods that can be cut and shaped into tablets, and films of a suitable thickness that shaped forms can be punched into suitable size and shape for administration. This extrudate may be used in further processing steps to yield the final pharmaceutical product or composition. The extrudate of the pharmaceutical composition may be dried, formed, milled, sieved, or any combination of these processes to obtain a final composition which may be administered to a patient. Such processes are routine and known in the art and include formulating the specific product to obtain a final pharmaceutical or nutraceutical product. Additionally, the extrudate of the pharmaceutical composition obtained may be processed using a tablet press to obtain a final tablet. Additionally, it may be milled and combined with one or more additional excipients to form a capsule or pressed into a tablet. The resultant pharmaceutical composition may also be dissolved in a solvent to obtain a syrup, a suspension, an emulsion, or a solution.

### IV. DEFINITIONS

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “active pharmaceutical ingredient”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, a reduction in the growth rate of cancer, or prevention of metastasis. Treatment of cancer may also refer to prolonging the survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide, and calcium hydroxide. Acceptable organic bases include ethanolamine, diethanolamine, triethanolamine, tromethamine, N-methylglucamine, and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified compound, wherein at least one of the compounds is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example, hydrochlorides, hydrobromides, sulfates, hydrogen sulfates or phosphates, salts with suitable carboxylic acids, such as optionally hydroxylated lower alkanoic acids, for example, acetic acid, glycolic acid, propionic acid, lactic acid or pivalic acid, optionally hydroxylated and/or oxo-substituted lower alkane dicarboxylic acids, for example, oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example, methanesulfonates, benzenesulfonates, p-toluenesulfonates or N-cyclohexylsulfamates (cyclamates).

The term “degradation” or “chemically sensitive” refers to a compound that is destroyed or rendered inactive and unacceptable for use. Degradation may include compounds which have one or more chemical bonds present in the compound has been broken.

The term “dissolution” as used herein refers to a process by which a solid substance, such as the active ingredients or one or more excipients, is dispersed in molecular form in a medium. The dissolution rate of the active ingredients of the pharmaceutical dose of the invention is defined by the amount of drug substance that goes in solution per unit time under standardized conditions of liquid/solid interface, temperature and solvent composition.

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active agent in the composition is measured by powder x-ray diffraction.

A “poorly soluble drug” refers to a drug which meets the requirements of the USP and BP solubility criteria of at least a sparingly soluble drug. The poorly soluble drug may be sparingly soluble, slightly soluble, very slightly soluble or practically insoluble. In a preferred embodiment, the drug is at least slightly soluble. In a more preferred embodiment, the drug is at least very slightly soluble. As defined by the USP and BP, a soluble drug is a drug which is dissolved from 10 to 30 part of solvent required per part of the solute, a sparingly soluble drug is a drug which is dissolved from 30 to 100 part of solvent required per part of the solute, a slightly soluble drug is a drug which is dissolved from 100 to 1,000 part of solvent required per part of the solute, a very slightly soluble drug is a drug which is dissolved from 1,000 to 10,000 part of solvent required per part of the solute, and a practically insoluble drug is a drug which is dissolved from 10,000 part of solvent required per part of solute. The solvent may be water that is at a pH from 1-7.5, preferably physiological pH.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significance such as “significantly”) is not meant to imply statistical differences between two values but only to imply importance or the scope of the difference of the parameter.

Throughout this application, the term “about” is used to indicate that a value includes the inherent variation of error for the device, the method being employed to determine the value or the variation that exists among the study subjects or experimental studies. Unless another definition is applicable, the term “about” refers to ±10% of the indicated value.

As used herein, the term “substantially free of” or “substantially free” in terms of a specified component, is used herein to mean that none of the specified components has been purposefully formulated into a composition and/or is present only as a contaminant or in trace amounts. The total amount of all containments, by-products, and other material is present in that composition in an amount of less than 2%. The term “essentially free of” or “essentially free” is used to represent that the composition contains less than 1% of the specific component. The term “entirely free of” or “entirely free” contains less than 0.1% of the specific component.

As used herein, the term “substantially intact” in terms of a specified component, is used herein to mean that the specified component has not been degraded or rendered inactive in an amount less than 5%. The term “essentially intact” is used to represent that less than 2% of the specific component has been degraded or rendered inactive. The term “entirely intact” contains less than 0.1% of the specific component that has been degraded or rendered inactive.

The term “homogenous” is used to mean a composition in which the components are mixed in such a way that the components are uniformly distributed amongst the composition. In a preferred embodiment, the composition is uniformly distributed in such a manner that there are no regions of a single component that are greater than 1 μm or more preferably less than 0.1 μm. In one embodiment, the composition is so homogeneously mixed in such a manner that there are no atoms of the electromagnetic energy absorbing excipients are adjacent to another atom of the electromagnetic energy absorbing excipients.

The terms “substantially” or “approximately” as used herein may be applied to modify any quantitative comparison, value, measurement, or other representation that could permissibly vary without resulting in a change in the basic function to which it is related.

A temperature, when used without any other modifier, refers to room temperature, preferably 23° C. unless otherwise noted. An elevated temperature is a temperature which is more than 5° C. greater than room temperature; preferably more than 10° C. greater than room temperature.

The term “unit dose” refers to a formulation of the pharmaceutical composition such that the formulation is prepared in a manner sufficient to provide a single therapeutically effective dose of the active agent to a patient in a single administration. Such unit dose formulations that may be used include but are not limited to a single tablet, capsule, or other oral formulations, or a single vial with a syringe able liquid or other injectable formulations. The resulting product can then undergo further downstream processing to create an intermediate product, such as granules, that can then be further formulated into a unit dose such as one prepared for oral delivery as tablets, capsules, three-dimensionally printed selective laser sintered (3DPSLS) or suspensions; pulmonary and nasal delivery; topical delivery as emulsions, ointments or creams; transdermal delivery; and parenteral delivery as suspensions, microemulsions or depot. In some forms, the final pharmaceutical composition that is produced is no longer a powder and is further produced as a homogenous final product. This final product has the capability of being processed into granules and being compressed or 3DPSLS into a final pharmaceutical unit dose form.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements and parameters.

Other objects, features, and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### V. EXAMPLES

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Methods and Materials

### A. Materials

Candurin© gold sheen was purchased from EMD Performance Materials (Philadelphia, PA). AQOAT® Hypromellose acetate succinate HMP grade (HPMCAS-HMP) was donated by Shin-Etsu Chemical Co., Ltd. (Tokyo, Japan). Boehringer Ingelheim (BI) research compound BI639667 (BI-667) was donated by BI (Ingelheim, Germany). Glass number 50 capillary (2.0 mm) was purchased from Hampton Research Corp. (Aliso Viejo, CA). HPLC grade acetonitrile, methanol and Trifluoracetic acid (TFA) were purchased from Fisher Scientific (Pittsburgh, PA). Monohydrate and dihydrate sodium phosphate salts were purchased from Fisher Scientific (Pittsburgh, PA). Fasted state simulated intestinal fluid (FaSSIF) powder was purchased from Biorelevant.com Ltd (Surrey, United Kingdom).

### B. Step 1—ASD Granule Manufacturing

A composition comprising a mixture of B1667, HPMCAS-HMP, and Candurin® was prepared with a ratio of components of 33:64:3. This composition was processed through a batch compounder at 2,500 RPM with an ejection temperature of 160° C. and a run time of 11 seconds. Thesse samples were then milled and sieved to obtain granules of less than 250 μm. These particles were then used in the SLS process.

### C. Step 2—SLS Processing of ASD Granules

The particles prepared in Step 1 were used in the SLS additive manufacturing process. To test the parameters, a composition with polymer and Candurin® was used. These samples were printed with the following printing conditions.

TABLE 1
Printing Conditions

| | Surface | Chamber | Laser | Hatch | |
|---|---|---|---|---|---|
| | Temperature | Temperature | Speed | Spacing | |
| | (° C.) | (° C.) | (mm/s) | (μm) | Comments |
| Example 1 | 115 | 100 | 100 | 25 | Trace |
| | | | | | Crystallinity |
| Example 2 | 115 | 100 | 100 | 5 | Amorphous |

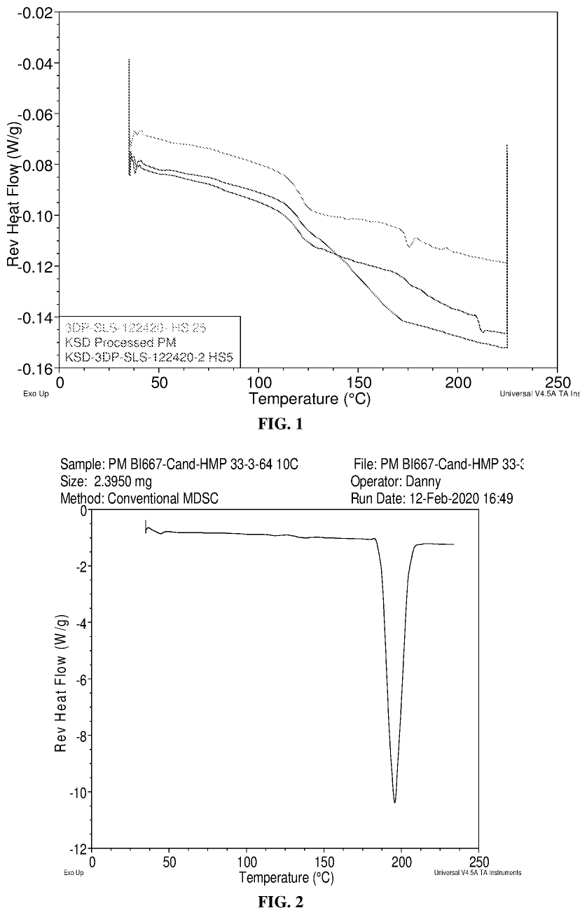

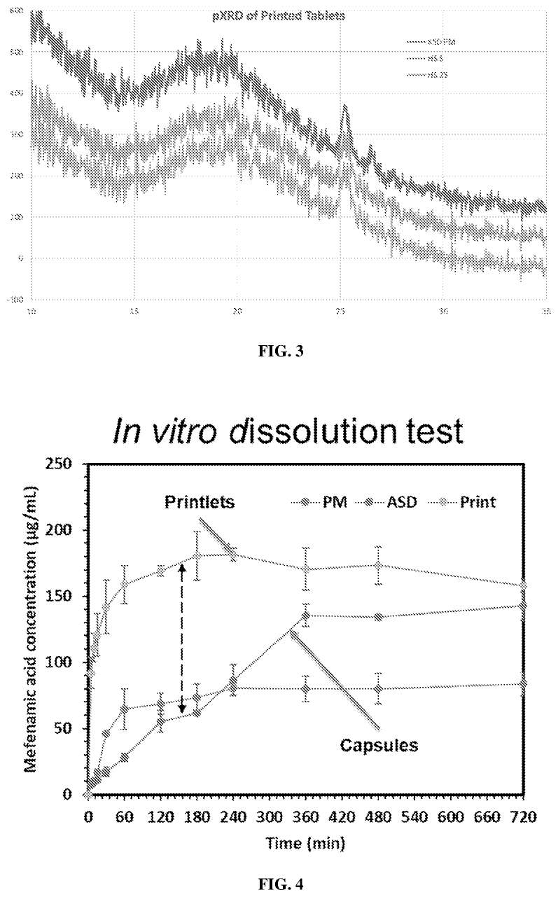

Printing parameters can lead to recrystallization of the amorphous active pharmaceutical ingredient. Examples with larger hatch spacings was more likely to lead to crystallization events. It is possible that the low hatch spacing examples lead to a potential phase separation suggesting that higher drug loading may be suitable for these methods. See FIGS. 1 & 2. Powder X-ray diffraction of both printing conditions and the physical mixture of the ASD showed little change in the crystallinity of the composition. See FIG. 3.

After these initial testing, an ASD was formulated with a PEO (polyethylene oxide) polymer with the ASD comprising 80% of the mixture measured as the weight over the weight of the entire composition. These formulations were prepared using the parameters describe din Table 2. These parameters included a hatch spacing of about 100 μm, chamber temperature of about 55° C., a surface temperature at room temperature, the laser surface temperature from about 105° C. to about 165° C., and a laser scanning speed from about 50 mm/s to about 1,000 mm/s. These compositions were analyzed via X-ray diffraction/WAXS, FT-IR, SEM, and DSC. In particular, the concentration of the active pharmaceutical ingredient after dissolution was carried out. The results are shown in FIG. 4. The printed ASD showed improved dissolution with a higher concentration compared to either the traditional ASD or the mere physical mixture.

TABLE 3
Printing Parameters ASD/PEO formulations

| | | Date | Trial | Power (%) | Speed | Spacing | Fluence | Temperature (mean, STD) | |
|---|---|---|---|---|---|---|---|---|---|
| | | Sep. 23, 2021 | 1.1 | 16 | 50 | 100 | 0.177 | 132.2 | 7.5 |
| | | Sep. 23, 2021 | 1.2 | 20 | 100 | 100 | 0.170 | 151.8 | 9.2 |
| Single | {open oversize brace} | Sep. 23, 2021 | 1.3 | 28 | 250 | 100 | 0.133 | 160.3 | 10.5 |
| Layer | | Sep. 23, 2021 | 1.4 | 28 | 500 | 100 | 0.067 | 137.1 | 7.4 |
| | | Sep. 23, 2021 | 1.5 | 35 | 1000 | 100 | 0.048 | 131 | 7 |
| | | Sep. 23, 2021 | 1.6 | 95 | 1000 | 100 | 0.170 | NaN | NaN |
| | | Sep. 23, 2021 | 2.1 | 16 | 50 | 100 | 0.177 | 133.1 | 7.9 |
| | | Sep. 23, 2021 | 2.2 | 20 | 100 | 100 | 0.170 | 148.6 | 9.3 |
| | | Sep. 23, 2021 | 2.3 | 22 | 250 | 100 | 0.084 | 123.8 | 6.5 |
| | | Sep. 23, 2021 | 2.4 | 28 | 500 | 100 | 0.067 | 134 | 7.1 |
| | | Sep. 23, 2021 | 2.5 | 35 | 1000 | 100 | 0.048 | 128.6 | 6.7 |
| Multiple | {open oversize brace} | Sep. 23, 2021 | 2.6 | 37 | 1000 | 100 | 0.052 | 138.1 | 7.5 |
| Layers | | Sep. 30, 2021 | 6.1 | 25 | 250 | 100 | 0.109 | 142.4 | 9 |
| | | Sep. 30, 2021 | 6.2 | 27 | 250 | 100 | 0.125 | 152.6 | 10.5 |
| | | Sep. 30, 2021 | 6.3 | 28 | 500 | 100 | 0.067 | 121.1 | 5.8 |
| | | Sep. 30, 2021 | 6.4 | 30 | 500 | 100 | 0.075 | 109.2 | 4.7 |
| | | Sep. 30, 2021 | 6.5 | 31 | 750 | 100 | 0.053 | 110.5 | 5.3 |
| | | Sep. 30, 2021 | 6.6 | 33 | 750 | 100 | 0.058 | 121 | 6.3 |

* * *

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the disclosure as defined by the appended claims.

### REFERENCES

The following references, to the extent that they provide exemplary procedural or other details supplementary to those set forth herein, are specifically incorporated herein by reference.
U.S. Patent App. Pub. No. 2019/037441
Baghel et al., Journal of Pharmaceutical Sciences, 105:2527-2544, 2016.
Czyrski and Sznura, Scientific Reports, 9:19458, 2019.
Damian et al., Journal of Optoelectronics and Advanced Materials, 9:780, 2007.
Davis et al., Journal of Pharmaceutical Sciences, S0022354920307413, 2020.
Fong et al., Expert Opinion on Drug Delivery, 14:403-426, 2017.
Handa et al., J Pharm Biomed Anal, 89:6-17, 2014.
Hayase et al. J Pharm Sci, 83:532-538, 1994.
Khuri and Mukhopadhyay, WIREs Computational Statistics. 2:128-149, 2010.
Lesaffre et al., Bayesian Methods in Pharmaceutical Research, CRC Press, 2020.
Majeed et al., Journal of Pharmacy and Pharmacology, 39:1044-1046, 1987.
Mantele and Deniz, Spectrochimica Acta Part A: Molecular and Biomolecular Spectroscopy 173:965-968, 2017.
Matsuda et al., International Journal of Pharmaceutics, 54:211-221, 1989.
Noordin et al., Journal of Materials Processing Technology, 145:46-58, 2004.
Politis et al., Drug Development and Industrial Pharmacy, 43:889-901, 2017.
Sabir et al., Journal of Microencapsulation, 0:1-16, 2021.
Sadana & Ghogare, International Journal of Pharmaceutics, 70:195-199, 1991.
Thakkar et al., International Journal of Pharmaceutics, 576:118989, 2020.
Weissman and Anderson, Org. Process Res. Dev., 19:1605-1633, 2015.
Wichianphong and Charoenchaitrakool, Journal of CO2 Utilization, 26:212-220, 2018.
Zhang et al., International Journal of Pharmaceutics 119945, 2020.

What is claimed is:

1. A method of preparing a pharmaceutical composition comprising:
(A) obtaining an amorphous solid dispersion comprising:
(1) an active pharmaceutical ingredient;
(2) a pharmaceutically acceptable polymer; and
(3) an excipient
(B) subjecting the amorphous solid dispersion to an additive manufacturing technique.

2. The method of claim 1, wherein the amorphous solid dispersion is prepared through a mixing process.

3. The method of claim 2, wherein the mixing process is either hot melt extrusion or a high energy fusion process.

4. The method according to any one of claims 1-3, wherein the amorphous solid dispersion is prepared using a mixing process with a processing speed from about 500 RPM to about 5,000 RPM.

5. The method of claim 4, wherein the processing speed is from about 1,000 RPM to about 4,000 RPM.

6. The method of either claim 4 or claim 5, wherein the processing speed is from about 2,000 RPM to about 3,000 RPM.

7. The method according to any one of claims 4-6, wherein the processing speed is about 2,500 RPM.

8. The method according to any one of claims 1-3, wherein the amorphous solid dispersion is prepared using a mixing process with a processing speed from about 5 RPM to about 100 RPM.

9. The method of claim 8, wherein the processing speed is from about 10 RPM to about 80 RPM.

10. The method of either claim 8 or claim 9, wherein the processing speed is from about 20 RPM to about 75 RPM.

11. The method according to any one of claims 1-10, wherein the amorphous solid dispersion is prepared through a mixing process with an ejection temperature from about 20° C. to about 250° C.

12. The method of claim 11, wherein the ejection temperature is from about 75° C. to about 225° C.

13. The method of either claim 11 or claim 12, wherein the ejection temperature is from about 100° C. to about 225° C.

14. The method according to any one of claims 11-13, wherein the ejection temperature is from about 150° C. to about 200° C.

15. The method according to any one of claims 11-14, wherein the ejection temperature is about 160° C.

16. The method according to any one of claims 1-15, wherein the amorphous solid dispersion is prepared through a mixing process with a run time from about 3 seconds to about 5 minutes.

17. The method of claim 16, wherein the run time is from about 5 seconds to about 3 minutes.

18. The method of either claim 16 or claim 17, wherein the run time is from about 10 seconds to about 2 minutes.

19. The method of claim 1, wherein the amorphous solid dispersion is prepared through a solvent evaporation process.

20. The method of either claim 1 or claim 19, wherein the solvent evaporation process is spray drying.

21. The method according to any one of claims 1-20, wherein the amorphous solid dispersion is milled.

22. The method according to any one of claims 1-21, wherein the amorphous solid dispersion is sieved.

23. The method according to any one of claims 1-22, wherein the amorphous solid dispersion is milled and sieved.

24. The method of either claim 22 or claim 23, wherein the amorphous solid dispersion is sieved to particle size of less than 1 mm.

25. The method of claim 24, wherein the particle size is less than 500 μm.

26. The method of either claim 24 or claim 25, wherein the particle size is less than 250 μm.

27. The method according to any one of claims 1-26, wherein the additive manufacturing technique is vat photopolymerization, material jetting, binder jetting, powder-bed fusion, material extrusion, directed energy deposition, or sheet lamination.

28. The method of claim 27, wherein the additive manufacturing technique is fused deposition modeling, binder spraying, or selective laser sintering.

29. The method according to any one of claims 1-28, wherein the additive manufacturing technique comprises exposing the composition to an energy source to form a pattern.

30. The method according to any one of claims 1-29, wherein the additive manufacturing technique comprises exposing the composition to an energy source and the energy source is insufficient to cause the composition to recrystallize.

31. The method of claim 30, wherein less than 10% of the composition has recrystallized.

32. The method of either claim 30 or claim 31, wherein less than 5% of the composition has recrystallized.

33. The method according to any one of claims 30-32, wherein less than 2% of the composition has recrystallized.

34. The method according to any one of claims 30-33, wherein less than 1% of the composition has recrystallized.

35. The method according to any one of claims 1-29, wherein the energy source has a hatch spacing from about 0.1 μm to about 250 μm.

36. The method of claim 35, wherein the hatch spacing is from about 1 μm to about 200 μm.

37. The method of claim 36, wherein the hatch spacing is from about 2.5 μm to about 150 μm.

38. The method of claim 37, wherein the hatch spacing is about 5 μm, 25 μm, or 100 μm.

39. The method according to any one of claims 1-38, wherein the method comprises exposing the composition to a laser in a pattern.

40. The method according to any one of claims 1-39, wherein the method comprises depositing a layer of the composition onto a surface in a chamber.

41. The method of claim 40, wherein the layer has a layer thickness from about 1 μm to about 5 mm.

42. The method of claim 41, wherein the layer thickness is from about 10 μm to about 2.5 mm.

43. The method of either claim 41 or claim 42, wherein the layer thickness is from about 50 μm to about 1 mm.

44. The method according to any one of claims 41-43, wherein the layer thickness is from 50 μm to about 400 μm.

45. The method according to any one of claims 40-44, wherein the layer comprises a surface temperature at its surface different from a chamber temperature in the chamber.

46. The method of claim 45, wherein the surface temperature is from about 0° C. to about 250° C.

47. The method of either claim 45 or claim 46, wherein the surface temperature is from about 50° C. to about 175° C.

48. The method according to any one of claims 45-47, wherein the surface temperature is from about 75° C. to about 150° C.

49. The method according to any one of claims 45-48, wherein the chamber temperature is from about 25° C. to about 250° C.

50. The method according to any one of claims 45-49, wherein the chamber temperature is from about 40° C. to about 200° C.

51. The method according to any one of claims 45-50, wherein the chamber temperature is from about 50° C. to about 150° C.

52. The method according to any one of claims 29-51, wherein the pattern is prepared by passing the energy source over the composition with a laser speed from about 0.1 mm/s to about 5,000 mm/s.

53. The method of claim 52, wherein the laser speed is from about 0.5 mm/s to about 2,500 mm/s.

54. The method of either claim 52 or claim 53, wherein the laser speed is from about 1 mm/s to about 2,000 mm/s.

55. The method according to any one of claims 52-54, wherein the laser speed is 100 mm/s.

56. The method according to any one of claims 29-55, wherein the energy source is a laser.

57. The method of claim 56, wherein the laser comprises a laser power from about 0.1 W to about 250 W.

58. The method of either claim 56 or claim 57, wherein the laser power is from about 5 mW to about 20 W.

59. The method according to any one of claims 56-58, wherein the laser power is from about 50 mW to about 1 W.

60. The method according to any one of claims 56-59, wherein the laser power is from about 100 mW to about 500 mW.

61. The method according to any one of claims 56-60, wherein the laser comprises a beam size from about 0.25 μm to about 1 mm.

62. The method of claim 61, wherein the beam size is from about 1 μm to about 500 μm.

63. The method of either claim 61 or claim 62, wherein the beam size is from about 2.5 μm to about 100 μm.

64. The method according to any one of claims 56-63, wherein the laser has a wavelength from about 50 nm to about 15,000 nm.

65. The method of claim 64, wherein the wavelength is from about 5 nm to about 11,000 nm.

66. The method of either claim 64 or claim 65, wherein the wavelength is from about 200 nm to about 1,000 nm.

67. The method according to any one of claims 56-66, wherein the laser gives the composition an amount of energy equal to an electron laser density from about 2.5 J/mm³ to about 500 J/mm³.

68. The method of claim 67, wherein the electron laser density is from about 5 J/mm³ to about 250 J/mm³.

69. The method of either claim 67 or claim 68, wherein the electron laser density is from about 7.5 J/mm³ to about 50 J/mm³.

70. The method according to any one of claims 67-69, wherein the electron laser density is greater than 2.5 J/mm³.

71. The method according to any one of claims 67-70, wherein the electron laser density is greater than 5 J/mm³.

72. The method according to any one of claims 67-71, wherein the electron laser density is greater than 7.5 J/mm³.

73. The method according to any one of claims 1-72, wherein the amorphous solid dispersion comprises at least 90% of the active pharmaceutical ingredient in the amorphous form.

74. The method of claim 73, wherein the amorphous solid dispersion comprises at least 95% of the active pharmaceutical ingredient in the amorphous form.

75. The method of either claim 73 or claim 74, wherein the pharmaceutical composition comprises at least 99% of the active pharmaceutical ingredient in the amorphous form.

76. The method according to any one of claims 1-75, wherein the active pharmaceutical ingredient and the pharmaceutically acceptable polymer is homogenously mixed together.

77. The method according to any one of claims 1-76, wherein the active pharmaceutical ingredient is a poorly soluble drug.

78. The method according to any one of claims 1-77, wherein the active pharmaceutical ingredient is a BCS class 2 drug.

79. The method according to any one of claims 1-77, wherein the active pharmaceutical ingredient is a BCS class 3 drug.

80. The method according to any one of claims 1-77, wherein the active pharmaceutical ingredient is a BCS class 4 drug.

81. The method according to any one of claims 1-80, wherein the active pharmaceutical ingredient is an agent which undergoes degradation at an elevated temperature in a formulation process.

82. The method according to any one of claims 1-81, wherein the active pharmaceutical ingredient is chemically sensitive to temperature.

83. The method according to any one of claims 1-82, wherein the active pharmaceutical ingredient is chemically sensitive to shear.

84. The method according to any one of claims 1-83, wherein the active pharmaceutical ingredient is an agent with a melting point of greater than 60° C.

85. The method of claim 84, wherein the melting point is from about 60° C. to about 300° C.

86. The method of claim 85, wherein the melting point is from about 80° C. to about 200° C.

87. The method according to any one of claims 1-86, wherein the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives.

88. The method according to any one of claims 1-87, wherein the amorphous solid dispersion comprises from about 1% w/w to about 90% w/w of the active pharmaceutical ingredient.

89. The method according to any one of claims 1-88, wherein the amorphous solid dispersion comprises from about 5% w/w to about 50% w/w of the active pharmaceutical ingredient.

90. The method according to any one of claims 1-89, wherein the amorphous solid dispersion comprises from about 10% w/w to about 30% w/w of the active pharmaceutical ingredient.

91. The method according to any one of claims 1-90, wherein the amorphous solid dispersion n comprises from about 5% w/w to about 30% w/w of the active pharmaceutical ingredient.

92. The method according to any one of claims 1-91, wherein the pharmaceutically acceptable polymer is a cellulosic polymer.

93. The method of claim 92, wherein the cellulosic polymer is a neutral cellulosic polymer.

94. The method of claim 92, wherein the cellulosic polymer is a charged cellulosic polymer.

95. The method according to any one of claims 1-91, wherein the pharmaceutically acceptable polymer is a neutral non-cellulosic polymer.

96. The method of claim 95, wherein the neutral non-cellulosic polymer comprises a poly(vinyl acetate), poly(vinylpyrrolidone), poly(ethylene glycol), poly(ethylene oxide), poly(vinyl alcohol), or methacrylate unit.

97. The method according to any one of claims 1-96, wherein the pharmaceutically acceptable polymer comprises a poly(vinyl acetate) or a methacrylate unit.

98. The method according to any one of claims 1-97, wherein the pharmaceutically acceptable polymer is a poly(vinyl acetate)-co-poly(vinylpyrrolidone) copolymer, dimethylaminoethyl methacrylate-methacrylic acid ester copolymer, ethylacrylate-methylmethacrylate copolymer, poly(vinyl acetate) phthalate, poly(methacrylate ethylacrylate) (1:1) copolymer, poly(methacrylate methylmethacrylate) (1:1) copolymer, poly(methacrylate methylmethacrylate) (1:2) copolymer, or polyvinyl caprolactam-polyvinyl acetate-polyethylene glycol graft copolymer sodium dodecyl sulfate.

99. The method according to any one of claims 1-98, wherein the amorphous solid dispersion comprises from about 5% w/w to about 95% w/w of the pharmaceutically acceptable polymer.

100. The method according to any one of claims 1-99, wherein the amorphous solid dispersion comprises from about 50% w/w to about 90% w/w of the pharmaceutically acceptable polymer.

101. The method according to any one of claims 1-100, wherein the amorphous solid dispersion comprises from about 60% w/w to about 90% w/w of the pharmaceutically acceptable polymer.

102. The method according to any one of claims 1-101, wherein the excipient is a material that leads to improved energy absorption.

103. The method according to any one of claims 1-102, wherein the excipient is a material with a lambda max (λₘₐₓ) equal to the wavelength of the laser.

104. The method of claim 103, wherein the lambda max is from about 50 nm to about 15,000 nm.

105. The method of either claim 103 or claim 104, wherein the lambda max is from about 200 nm to about 11,000 nm.

106. The method according to any one of claims 103-105, wherein the lambda max is from about 200 nm to about 1,000 nm.

107. The method according to any one of claims 1-106, wherein the excipient is an inorganic material.

108. The method according to any one of claims 1-107, wherein the excipient is an aluminum material.

109. The method of claim 108, wherein the aluminum material is an aluminum inorganic salt.

110. The method of claim 109, wherein the aluminum inorganic salt is bentonite, potassium aluminum silicate, aluminum, aluminum sulfates, sodium aluminum phosphate acidic, sodium aluminum silicate, calcium aluminum silicate, starch aluminum octenyl succinate, or potassium aluminum silicate with a coating of titanium dioxide and/or iron oxide.

111. The method of either claim 109 or claim 110, wherein the aluminum inorganic salt is potassium aluminum silicate with a coating of titanium dioxide and/or iron oxide.

112. The method of claim 107, wherein the inorganic material is iron oxide, titanium oxide, or silicates.

113. The method according to any one of claims 1-106, wherein the excipient is an organic material.

114. The method of claim 113, wherein the organic material is a dye.

115. The method of either claim 113 or claim 114, wherein the dye is carmine, a phthalocyanine, or a diazo compound.

116. The method according to any one of claims 1-115, wherein the amorphous solid dispersion comprises from about 0.01% w/w to about 60% w/w of the excipient.

117. The method according to any one of claims 1-116, wherein the amorphous solid dispersion comprises from about 0.1% w/w to about 50% w/w of the excipient.

118. The method according to any one of claims 1-117, wherein the amorphous solid dispersion comprises from about 1% w/w to about 30% w/w of the excipient.

119. The method according to any one of claims 1-118, wherein the amorphous solid dispersion comprises from about 1% w/w to about 10% w/w of the excipient.

120. The method according to any one of claims 1-119, wherein the amorphous solid dispersion further comprises one or more excipients.

121. The method according to any one of claims 1-120, wherein the excipient is a processing aid.

122. The method of claim 120 or claim 121, wherein the excipient is an opacifying agent.

123. The method according to any one of claims 1-120, wherein the amorphous solid dispersion comprises a flowability excipient.

124. The method according to any one of claims 1-123, wherein the flowability excipient is a silicon compound.

125. The method according to any one of claims 1-124, wherein the flowability excipient is silicon dioxide.

126. The method according to any one of claims 1-125, wherein the amorphous solid dispersion comprises from about 0.1% w/w to about 5% w/w of the flowability excipient.

127. The method of claim 126, wherein the amorphous solid dispersion comprises from about 0.5% w/w to about 2.5% w/w of the flowability excipient.

128. The method of either claim 126 or claim 127, wherein the amorphous solid dispersion comprises from about 0.5% w/w to about 1.5% w/w of the flowability excipient.

129. A pharmaceutical composition prepared according to the methods according to any one of claims 1-128.

130. A method of treating or preventing a disease or disorder in a patient comprising administering to the patient in need thereof a therapeutically effective amount of a composition prepared according to the methods of any one of claims 1-129; wherein the active pharmaceutical ingredient in the composition is sufficient to treat or prevent the disease or disorder.

131. Use of a composition prepared according to the method of any one of claims 1-129 for the treatment or prevention of a disease or disorder in a patient; wherein the active pharmaceutical ingredient in the composition is sufficient to treat or prevent the disease or disorder.

132. A composition for use in the treatment or prevention of a disease or disorder in a patient comprising a therapeutically effective amount of a composition prepared according to the methods of any one of claims 1-129; wherein the active pharmaceutical ingredient in the composition is sufficient to treat or prevent the disease or disorder.
